(19) United States
(12) Patent Application Publication
(10) Pub. No.: US 2025/0186754 A1
(43) Pub. Date: Jun. 12, 2025

(54) Compliant Polymeric Structures Having Polymeric Microneedles and Methods for Making and Using Same

(71) Applicant: The Board of Trustees of the Leland Stanford Junior University, Stanford, CA (US)

(72) Inventors: Joseph M. DeSimone, Stanford, CA (US); Cooper Owen Shea, Stanford, CA (US); Dan Ilyin, Stanford, CA (US); Gunilla B. Jacobson, Stanford, CA (US); Jean Won Kwak, Stanford, CA (US); Kaiwen Hsiao, Stanford, CA (US); Maria T. Dulay, Stanford, CA (US); Madison M. Driskill, Stanford, CA (US); Micah Lawrence, Stanford, CA (US); Netra Rajesh, Stanford, CA (US); Olayemi Oluwatosin Ajao, Stanford, CA (US)

(21) Appl. No.: 18/846,097

(22) PCT Filed: Apr. 20, 2023
(86) PCT No.: PCT/US2023/019276

§ 371 Date: Sep. 11, 2024

Related U.S. Application Data
(60) Provisional application No. 63/333,655, filed on Apr. 22, 2022.

(51) Int. Cl. A61M 37/00

(52) U.S. Cl. CPC A61M 37/0015; A61M 2037/0023; A61M 2037/0046; A61M 2037/0053; A61M 2037/0061; A61M 2202/0007; A61M 2205/0216; A61M 2205/0244; A61M 2207/00

(57) ABSTRACT

Aspects of the present disclosure include polymeric structures having one or more polymeric microneedles. Polymeric structures according to certain embodiments exhibit a macrostructural change (e.g., exhibit elastic deformation) in response to an applied stimulus. In some embodiments. polymeric structures include a microarray of polymeric microneedles for delivering an active agent compound to a subject or for collecting a biological fluid sample from a subject. Methods for applying a polymeric structure having polymeric microneedles to a skin surface of a subject is also described. Methods for making the polymeric structures, such as by high resolution continuous liquid interface production is also provided. Kits having one or more of the subject polymeric structures are also described.

### CROSS-REFERENCE TO RELATED APPLICATIONS

Pursuant to 35 U.S.C. § 119(e), this application claims priority to the filing date of U.S. Provisional Patent Application Ser. No. 63/333,655 filed Apr. 22, 2022; the disclosure of which application is incorporated herein by reference in its entirety.

### INTRODUCTION

The intradermal (ID) space has been actively explored as a means for drug delivery and diagnostics that are minimally invasive. Intradermal drug delivery is the process of delivering formulations into layers of skin. ID access necessitates puncturing the outermost layer of skin called the stratum corneum (StC), a tough barrier that provides mechanical integrity for the skin. Human skin is a complex, multi-layer organ, that includes the stratum corneum, epidermis, dermis and hypodermis. Often, these treatments target either the epidermal or dermal layers of skin, which are situated above blood vessels and nerve fibers of the skin. It offers an attractive alternative to intravenous (IV) injection, which often elicits systemic effects and can be particularly advantageous for targeted, local drug delivery. ID drug delivery can provide for the ability to deliver compounds with a significant first-pass effect, or metabolization by the liver which can prematurely degrade the therapeutic compound, upon systemic administration. Further, ID access also reduces pain associated with hypodermic injections and can help eliminate the risk of transmitting blood-borne diseases through the generation of dangerous medical waste. ID access can also be self-administered and can eliminate reliance on trained medical professionals.

Microneedles or microneedle patches or Micro-Array Patches (MAPs) have a series of micrometer-sized projections that can painlessly puncture the skin and access the epidermal/dermal layer and facilitate sampling of interstitial fluid. MAPs are employed in cosmetics, such as for use in treating acne scars and stretch marks by penetrating the stratum corneum to create micro conduits that stimulate growth factor secretion and collagen production. Microneedles are conventionally solid or hollow microneedles that are micro-molded from templates and fabricated by a three-step process master fabrication, mold fabrication and mold filling to generate hollow, metallic projections with uniform geometries. Microneedles have been generally manufactured to be produced in a manner like conventional hypodermic needles.

### SUMMARY

Aspects of the present disclosure include polymeric structures having one or more polymeric microneedles. Polymeric structures according to certain embodiments exhibit a macrostructural change (e.g., exhibit elastic deformation) in response to an applied stimulus. In some embodiments, polymeric structures include a microarray of polymeric microneedles for delivering an active agent compound to a subject or for collecting a biological fluid sample from a subject. Methods for applying a polymeric structure having polymeric microneedles to a skin surface of a subject is also described. Methods for making the polymeric structures, such as by high resolution continuous liquid interface production is also provided. Kits having one or more of the subject polymeric structures are also described.

In embodiments, polymeric structures described herein are dynamic structures which can alter geometry or shape in response to an applied stimulus (e.g., pressure manually applied when contacting with a skin surface of a subject). In some instances, the dynamic polymeric structure is compliant and exhibits motion through elastic deformation of the polymeric microstructure. In some instances, the polymeric structure is configured to change shape in response to the applied stimulus. In some instances, the change in shape is reversible. In other instances, the change in shape is irreversible. In some instances, the polymeric structure includes one or more hinges that are configured to extend laterally in response to the applied stimulus. In some instances, the polymeric structure includes a housing for each of the polymeric microneedles, where the housing has a kerf bend which expands laterally in response to applied stimulus. In certain embodiments, the polymeric structure is configured to change size in response to the applied stimulus. The change in size may be reversible or irreversible. In some instances, the polymeric structure compresses in response to the applied stimulus. In some instances, the polymeric structure expands in response to the applied stimulus. In some embodiments, the polymeric structure includes a plurality of polymeric microneedles, such as where the polymeric structure includes an array of polymeric microneedles.

In some embodiments, the polymeric structure includes a substrate having one or more polymeric microneedles and an alignment component. In some instances, one or more of the substrate and the alignment component has an aligner. In some instances, the aligner is a notch, a groove or a hole. In other instances, the aligner is a protrusion, such as a polygonal-shaped protrusion. In certain instances, the aligner is a cantilever hook. In some embodiments, the polymeric structure is configured to have locking and unlocking mechanisms, such as where the substrate is configured to reversibly or irreversibly coupled to the alignment component. In certain instances, coupling the substrate to the alignment component is sufficient to position the polymeric structure are a desired height on the skin surface of a subject. In some embodiments, the alignment component includes one or more holes which when coupled to the substrate (e.g., locked in place) is configured for passing the polymeric microneedles therethrough.

In some embodiments, one or more of the polymeric microneedles is configured to deploy a projection in response to the applied stimulus. For example, when the polymeric structure is pressed to the skin surface of a subject, a protrusion may be deployed from the microneedle. In some embodiments, the protrusion functions to maintain the polymeric microneedle in place beneath the skin surface of the subject. In some instances, the projection is a barb. In some instances, the projection is a cantilever hook. In certain instances, the projection is retractable, such as when the polymeric structure is to be removed from the skin surface of the subject. In certain embodiments, one or more of the polymeric microneedles is umbrella-shaped such that when a stimulus is applied, a plurality (e.g., 4 or more) protrusions are deployed which can be used to maintain the polymeric microneedle in place as well as to deliver an active agent compound beneath the skin surface of the subject. In certain instances, the umbrella-shaped polymeric microneedles are configured to collect biological fluid (e.g., interstitial or dermal fluid) from the subject and retracting the protrusions closes the microneedles in order to retain the collected biological fluid within the microneedle when the polymeric structure is removed from the skin of the subject.

In some embodiments, polymeric structures include a reservoir in fluid communication with the polymeric microneedles. In some instances, the reservoir is configured to contain an active agent compound (as described in greater detail below) to be delivered transdermally to a subject. In other instances, the reservoir is configured for collecting a biological fluid from a subject, such as where interstitial or dermal fluid collected from beneath a skin surface of a subject is conveyed to the reservoir through the polymeric microneedles. In some instances, the polymeric structure includes a pump component. In certain instances, the pump is configured to draw a fluidic medium from the polymeric microneedles into the reservoir. In other instances, the pump is configured to convey a fluidic medium from the reservoir through the polymeric microneedles.

In some embodiments, polymeric structures have lattice microstructures having lattice cell units. In some instances, the polymeric microneedle component of the polymeric structure have lattice microstructures having lattice cell units. In some embodiments, the lattice microstructures of the polymeric structures described herein have 2 or more repeating lattice cell units, such as 5 or more repeating lattice cell units. In certain embodiments, the lattice microstructure has a gradient in the lattice cell units such that the density of lattice cell units increases across a longitudinal axis of the polymeric structure. In some embodiments, the lattice cell unit has a lattice shape that is tetrahedral, Kagome, rhombic, icosahedral, Voronoi or triangular. In embodiments, the lattice cells have a unit size of from 10 μm to 1000 μm, such as from 200 μm to 500 μm. In some embodiments, the lattice microstructure includes a plurality of struts. In some instances, the struts have a thickness of from 25 μm to 150 μm, such as from 50 μm to 100 μm, for example 70 μm to 90 μm. Polymeric structures having a lattice microstructure of interest may have a length of from 500 μm to 2000 μm, such as from 700 μm to 1200 μm. In some embodiments, the lattice microstructure has a volume of from 0.01 μL to 2 μL. In embodiments, the polymeric structure is formed from a polymerizable material such as polycaprolactone, polyglycolic acid, polylactic acid, polylactic-co-glycolic acid, polyethylene glycol, thiol-enes, anhydrides, polyacrylic acid, poly methylmethacrylate, polyvinyl alcohol, polyvinylpyrrolidone, vinyl carbonates, vinyl esters, acrylamides, hyaluronic acid, chitosan, collagen, gelatin, carboxymethylcellulose, and blends or copolymers thereof. In certain embodiments, the polymeric structure is formed from polyethylene glycol dimethacrylate (PEGDMA). In some instances, the polymerizable material is biodegradable. In some instances, the polymeric structure is dissolvable in an aqueous medium, such as when applied intradermally to a subject.

In certain embodiments, polymeric microneedles of the polymeric structure also include an active agent compound. In some instances, the active agent compound is coated onto one or more surfaces of each of the microneedles. In some instances, the active agent compound is coated onto a tip section of each of the microneedles. In some instances, the active agent compound is coated onto a body section of each of the microneedles. In some instances, the active agent compound is coated onto a base section of each of the microneedles. In some embodiments, the active agent compound is contained within the lattice microstructure of the polymeric microneedle. In some embodiments, the active agent compound fills 1% or more of the void volume of the lattice microstructure, such as 10% or more, such as 25% or more and including 50% or more of the void volume of the lattice microstructure. In some embodiments, each polymeric microneedle contains 0.01 μL or more of the active agent, such as 0.05 μL or more and including 0.1 μL or more of the active agent. The active agent compound in some instances is a small molecule active agent. In other instances, the active agent is an immunogenic active agent, such as a vaccine.

Aspects of the present disclosure also include methods for applying the polymeric structures having a plurality of polymeric microneedles to a skin surface of a subject. In some instances, the polymeric structure includes microneedles arranged in an array on a substrate. In some instances, the polymeric structure further includes a backing layer (e.g., having a pressure sensitive adhesive). In certain instances, the polymeric structure is applied to the skin surface of the subject and maintained in contact with the subject for an extended period of time, such as for 30 minutes or longer, such as 1 hour or longer and including for 6 hours or longer. In certain instances, the patch is applied to the skin surface of the subject and removed within 15 minutes or less, such as within 5 minutes or less and including within 1 minute or less.

In some embodiments, methods include applying the polymeric structure to deliver a therapeutically effective amount of an active agent compound to the subject. In these embodiments, the plurality of polymeric microneedles contain an active agent compound and the polymeric structure is maintained in contact with the subject for a period of time sufficient to deliver one or more doses of the active agent compounds, such 2 or more doses and include 5 or more doses. In certain cases, some instances, the polymeric structure is maintained in contact with the subject for sustained release of the active agent to the subject over a period of time. In some instances, methods include applying the polymeric structure to the skin surface of the subject in a manner sufficient to collect a biological fluid sample from the subject into the microneedles. In some embodiments, methods include collecting interstitial fluid from the subject into the microneedles. In other embodiments, methods include collecting dermal fluid from the subject into the microneedles. Methods according to certain instances, include collecting 0.01 μL to 250 μL of the biological fluid from the subject, such as from 0.01 μL to 2 μL. In some embodiments, methods include collecting a biological fluid sample from the subject (e.g., interstitial fluid, dermal fluid) for detecting an analyte present in the biological sample, such as for detecting glucose.

Aspects of the disclosure also include methods for making a polymeric structure having one or more polymeric microneedles that is configured to exhibit a macrostructural change in response to an applied stimulus. Methods according to certain embodiments, include irradiating a polymerizable composition positioned between a build elevator and a build surface to generate a polymerizable composition having a first polymerized region of the polymerizable composition in contact with the build elevator and a first non-polymerized region of the polymerizable composition in contact with the build surface; displacing the build elevator away from the build surface; irradiating the first non-polymerized region of the polymerizable composition to generate a second polymerized region of the polymerizable composition in contact with the first polymerized region and a second non-polymerized region in contact with the build surface and repeating in a manner sufficient to generate the polymeric structure. In some embodiments, the polymerizable composition is in contact with the build elevator and the build surface. In some instances, methods include irradiating the polymerizable composition for a duration sufficient to bond the first polymerized region of the polymerizable composition to the build elevator. In some instances, the build elevator is displaced in predetermined increments of from 0.5 μm to 1.0 μm. In certain instances, polymerizable composition is added to the build surface after each displacement of the build elevator away from the build surface. In some embodiments, the polymerizable composition is irradiated through build surface. In some instances, the polymerizable composition is irradiated in the presence of a polymerization inhibitor. In certain embodiments, the polymerizable composition is continuously polymerized while displacing the build elevator away from the build surface. In certain cases, the polymerization inhibitor is oxygen and the build surface is permeable to oxygen.

In some embodiments, methods include preparing a polymeric structure having polymeric microneedles that include an active agent compound. In some instances, the active agent compound is coated onto a surface of the polymeric microneedles. In some embodiments, the active agent compound is coated onto the surface of the polymeric microneedle by dip-coating or by spray coating. In other instances, the active agent compound is dry-cast (e.g., as a powder) onto the surface of the polymeric microneedles. In some instances, the active agent compound is incorporated into an interior space of the polymeric microneedles. In some instances, the active agent is injected into the interior space of the polymeric microneedles (e.g., the lattice microstructure). In other instances, the active agent is introduced into the polymeric microneedle by contacting the lattice microstructure with a composition containing the active agent compound and incorporating the active agent by capillary action. In yet other instances, the active agent compound is incorporated into the polymerizable composition and is incorporated within the interior space of the polymeric microneedle while forming the lattice microstructure.

### BRIEF DESCRIPTION OF THE FIGURES

The invention may be best understood from the following detailed description when read in conjunction with the accompanying drawings. Included in the drawings are the following figures:

FIG. 1 depicts a polymeric structure having hinges that are configured to exhibit motion due to elastic deformation in response to an applied stimulus according to certain embodiments.

FIG. 2 depicts a polymeric structure having where the polymeric microneedle includes a housing component according to certain embodiments.

FIG. 3 depicts a polymeric structure that includes a substrate having polymeric microneedles and an alignment component according to certain embodiments.

FIG. 4A depicts a polymeric structure having a polymeric microneedle that includes a protrusion that is deployed in response to an applied stimulus according to certain embodiments. FIG. 4B depicts a polymeric structure having a polymeric microneedle that includes protrusions which extend in an umbrella-like mechanism in response to an applied stimulus according to certain embodiments. FIG. 4C depicts a polymeric structure having a plurality of polymeric microneedles which deploy protrusions in response to applied stimulus according to certain embodiments. FIG. 4D depicts pictures of polymeric structures having polymeric microneedles which deploy a plurality of protrusions generated by high resolution continuous liquid interface production according to certain embodiments.

FIG. 5 depicts a polymeric structure having a pump and reservoir component according to certain embodiments.

FIG. 6 depicts a microarray of polymeric microneedles which exhibit flexibility in response to applied pressure according to certain embodiments.

FIG. 7A depicts polymeric microneedles having a solid active agent compound according to certain embodiments. FIG. 7B depicts polymeric microneedles having a liquid active agent compound according to certain embodiments. FIG. 7C depicts capillary action by lattice microstructures according to certain embodiments.

### DETAILED DESCRIPTION

Aspects of the present disclosure include polymeric structures having one or more polymeric microneedles. Polymeric structures according to certain embodiments exhibit a macrostructural change (e.g., exhibit elastic deformation) in response to an applied stimulus. In some embodiments, polymeric structures include a microarray of polymeric microneedles for delivering an active agent compound to a subject or for collecting a biological fluid sample from a subject. Methods for applying a polymeric structure having polymeric microneedles to a skin surface of a subject is also described. Methods for making the polymeric structures, such as by high resolution continuous liquid interface production is also provided. Kits having one or more of the subject polymeric structures are also described.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

As summarized above, the present disclosure provides polymeric structures having one or more polymeric microneedles which exhibit a macrostructural change in response to an applied stimulus. In further describing embodiments of the disclosure, compliant polymeric structures are first described in greater detail. Next, methods for applying a polymeric structure to a skin surface of a subject, such as to deliver an active agent compound or to collect a biological fluid sample from the subject are described. Methods for making the polymeric structures, such as by high resolution continuous liquid interface production is also provided. Kits having one or more of the subject polymeric structures are then described.

### Compliant Polymeric Structures Having Polymeric Microneedles

Aspects of the present disclosure include polymeric structures having one or more polymeric microneedles configured to exhibit a macrostructural change in response to an applied stimulus. As described in detail herein, a macrostructural change refers to a change in the physical structure of the polymeric structure, such as the shape or a physical dimension. In some instances, polymeric structures described herein are dynamic structures that are compliant and exhibit motion through elastic deformation of the polymeric microstructure. The deformation of the polymeric structure in response to the applied stimulus varies depending on the intensity (e.g., amount of applied mechanical pressure) of the stimulus, where the polymeric structure may be deformed by 1% or more in any given dimension, such by 2% or more, such as by 5% or more, such as by 10% or more and including by 25% or more. In some instances, polymeric structures include a rigid component which does not change shape or size when the stimulus is applied and a compliant component which is configured to change shape or size in response to the applied stimulus.

In some embodiments, the macrostructural change such as deformation of the polymeric structure is irreversible. In some instances, the macrostructural change is reversible. In other instances, the macrostructural change is partially reversible, such as where shape or size of the polymeric structure reverts back to 50% or more of the original shape or size before the stimulus was applied, such as 60% or more, such as 70% or more, such as 80% or more, such as 90% or more and including where the shape or size of the polymeric structure reverts back to 95% or more of the original shape or size before the stimulus was applied. The stimulus applied to the polymeric structure may be a mechanical stimulus, such as manual pressure applied to the polymeric structure when inserting the polymeric microneedles into the skin surface of a subject (as described in greater detail below).

In some instances, the polymeric structure is configured to change shape in response to the applied stimulus. In some instances, the polymeric structure exhibits motion in response to elastic deformation when the stimulus is applied to the polymeric structure. The motion exhibited by the polymeric structure may be in any dimension, e.g., along an XY plane, along a YZ plane, along an XZ plane or a combination thereof. In some instances, deformation of the polymeric structure provides for motion of 0.001 mm or more, such as 0.005 mm or more, such as 0.01 mm or more, such as 0.05 mm or more, such as 0.1 mm or more, such as 0.5 mm or more, such as 1 mm or more, such as 2 mm or more, such as 3 mm or more, such as 4 mm or more, such as 5 mm or more, such as 10 mm or more, such as 15 mm or more, such as 20 mm or more and including by 50 mm or more. In some instances, the polymeric structure includes one or more hinges that are configured to extend laterally (along an X-axis where pressure is applied along the Y-axis) in response to the applied stimulus. In some instances, the hinges are rigid and the applied stimulus provides for lateral motion or 0.001 mm or more, such as 0.005 mm or more, such as 0.01 mm or more, such as 0.05 mm or more, such as 0.1 mm or more, such as 0.5 mm or more, such as 1 mm or more, such as 2 mm or more, such as 3 mm or more, such as 4 mm or more, such as 5 mm or more, such as 10 mm or more, such as 15 mm or more, such as 20 mm or more and including by 50 mm or more. As described in greater detail below, the hinges may be configured to contact the skin surface of a subject and downward pressure applied to the polymeric structure to insert the polymeric microneedles into the skin is sufficient extend the hinges to laterally stretch the skin that is adjacent to the insertion region of the microneedles. In other words, the dynamic motion of the hinges caused by the applied pressure during insertion of the microneedles stretches the skin surface, which in certain instances, facilitates easier and less painful microneedle insertion.

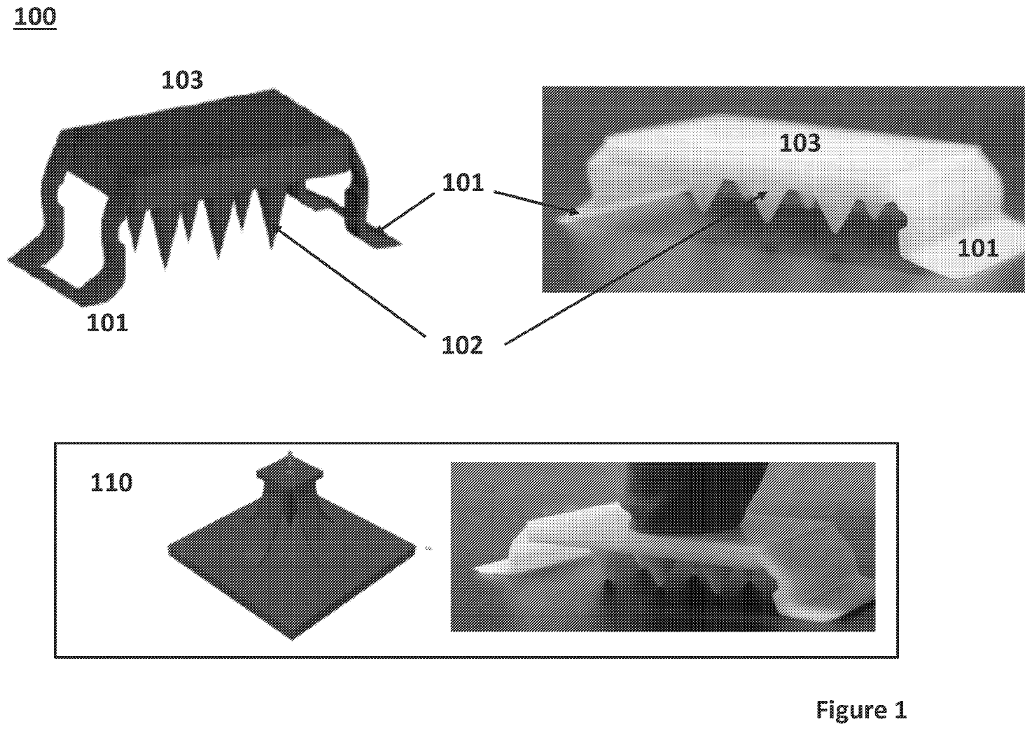

FIG. 1 depicts a polymeric structure having hinges that are configured to exhibit motion due to elastic deformation in response to an applied stimulus according to certain embodiments. Polymeric structure 100 includes hinges 101 and polymeric microneedles 102 where pressure applied to surface 103 causes hinges 101 to extend laterally. In embodiments, polymeric structure 100 may be pressed against a skin surface of a subject to insert microneedles 102 into the skin surface. When pressure is applied to surface 103, the lateral extension of hinges 101 provide for skin stretching to facilitate insertion of microneedles 102 into the skin. As depicted in inset 110 the hinges exhibit motion due to elastic formation when pressure is applied from above which stretches the skin to facilitate needle insertion.

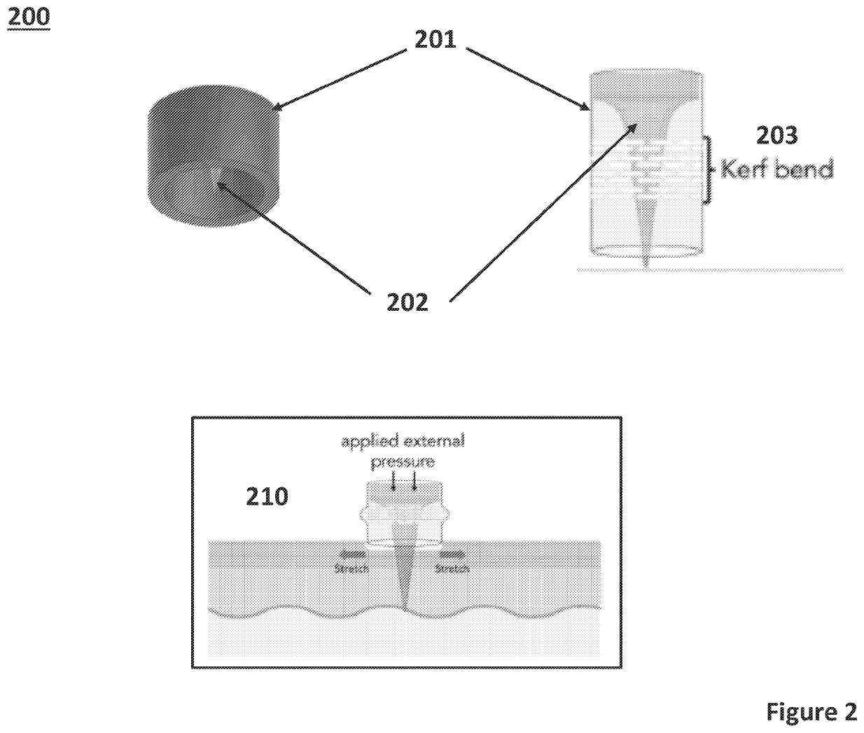

In some embodiments, the polymeric structure includes a housing for one or more of the polymeric microneedles, such as where each polymeric microneedle includes a polymeric housing component which is configured to change shape in response to the applied stimulus. In certain instances, the housing includes a kerf bend such that applying pressure to the polymeric structure to insert the microneedle into the skin surface of the subject provides for compression of the housing. In some instances, the housing component of the polymeric structure is configured to compress by 0.0001 mm or more, such as by 0.0005 mm or more, such as by 0.001 mm or more, such as by 0.005 mm or more, such as by 0.01 mm or more, such as by 0.05 mm or more, such as by 0.1 mm or more, such as by 0.5 mm or more, such as by 1 mm or more, such as by 2 mm or more, such as by 3 mm or more, such as by 4 mm or more and including by 5 mm or more. In some instances, applying pressure to the polymeric structure laterally expands a part of the housing component, such as where a part of the housing expands by 0.0005 mm or more, such as by 0.001 mm or more, such as by 0.005 mm or more, such as by 0.01 mm or more, such as by 0.05 mm or more, such as by 0.1 mm or more, such as by 0.5 mm or more, such as by 1 mm or more, such as by 2 mm or more. such as by 3 mm or more, such as by 4 mm or more and including by 5 mm or more. FIG. 2 depicts a polymeric structure having where the polymeric microneedle includes a housing component according to certain embodiments. Polymeric structure 200 includes a polymeric microneedle 202 enclosed by housing 201. Housing 201 includes a kerf bend 203 which when pressure is applied to insert the microneedle into the skin surface causes lateral expansion of the housing at the kerf bend. As shown in inset 210 in certain embodiments the lateral expansion of the housing provides for pinching of the skin surface when the polymeric structure is pressed against the surface of the skin of the subject. By applying pressure from above to insert the microneedle into the skin surface, the housing expands laterally pinching and stretching the skin to better facilitate painless microneedle entry.

In some embodiments, the polymeric structure includes a substrate having one or more polymeric microneedles and an alignment component. In some instances, one or more of the substrate and the alignment component has an aligner, such as 2 or more aligners, such as 3 or more aligners and including 4 or more aligners. In some instances, one or more of the aligners is a notch. In some instances, one or more of the aligners is a groove. In some instances, one or more of the aligners is a hole. In other instances, one or more of the aligners is a protrusion. In some instances, the aligner is a polygonal-shaped protrusion. In certain instances, the aligner is a cantilever hook.

In some embodiments, the polymeric structure is configured to have locking and unlocking mechanisms, such as where the substrate is configured to reversibly or irreversibly coupled to the alignment component. In some instances, the substrate is configured to be coupled to the alignment component by a snap-in mechanism where an aligner on the substrate is coupled to an aligner on the alignment component. In one example, the substrate may include a notch that couples to a protrusion on the alignment component. In another example, the substrate may include a protrusion that couples to a hole in the alignment component. In certain instances, the substrate and alignment component include one or more magnets for coupling to substrate to the alignment component. In some embodiments, aligners of the substrate are reversibly coupled to the aligners of the alignment component (i.e., the aligners can be locked together and unlocked as desired). In other embodiments, the aligners of the substrate are configured to be irreversibly coupled to the aligners of the alignment component (i.e., the substrate and alignment component are permanently coupled together once they are locked into place with each other). In certain instances, coupling the substrate to the alignment component is sufficient to position the polymeric structure are a desired height on the skin surface of a subject. In some embodiments, the alignment component includes one or more holes which when coupled to the substrate (e.g., locked in place) is configured for passing the polymeric microneedles therethrough.

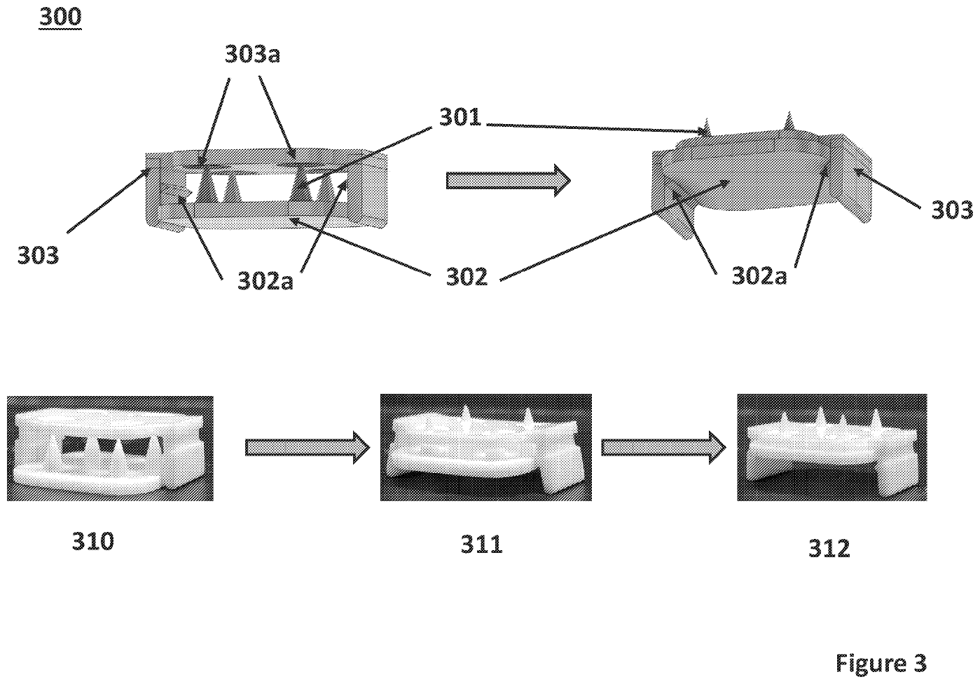

FIG. 3 depicts a polymeric structure that includes a substrate having polymeric microneedles and an alignment component according to certain embodiments. Polymeric structure 300 includes a substrate 302 having polymeric microneedles 301 positioned thereon. Alignment component 303 includes protrusions 302a for locking the substrate into a predetermined position when pressure is applied to the substrate, such as when inserting the microneedles into the skin surface of the subject. Alignment component 303 includes holes 303a which pass polymeric microneedles 301 when substrate 302 is locked into position past protrusions 302a. In some instances, polymeric microneedles are inserted into the skin of a subject by pressing substrate 302 through holes 303a and are locked in place to maintain the position of the polymeric microneedles in the skin of the subject with protrusions 302a. Inset 310 depicts the substrate coupled to alignment component 303 in an unlocked position. Applying mechanical pressure to polymeric structure 300 is sufficient to move substrate 302 as shown in inset 311 such that polymeric microneedles 301 begin to pass through holes 303a on alignment component 303. Further mechanical pressure applied to the substrate moves substrate 302 past protrusions 302a and locks polymeric microneedles 301 in place, such as within the skin of a subject. The substrate 302 can be moved back past protrusions 302a to unlock the substrate and retract polymeric microneedles 301 through holes 303a on alignment component 303.

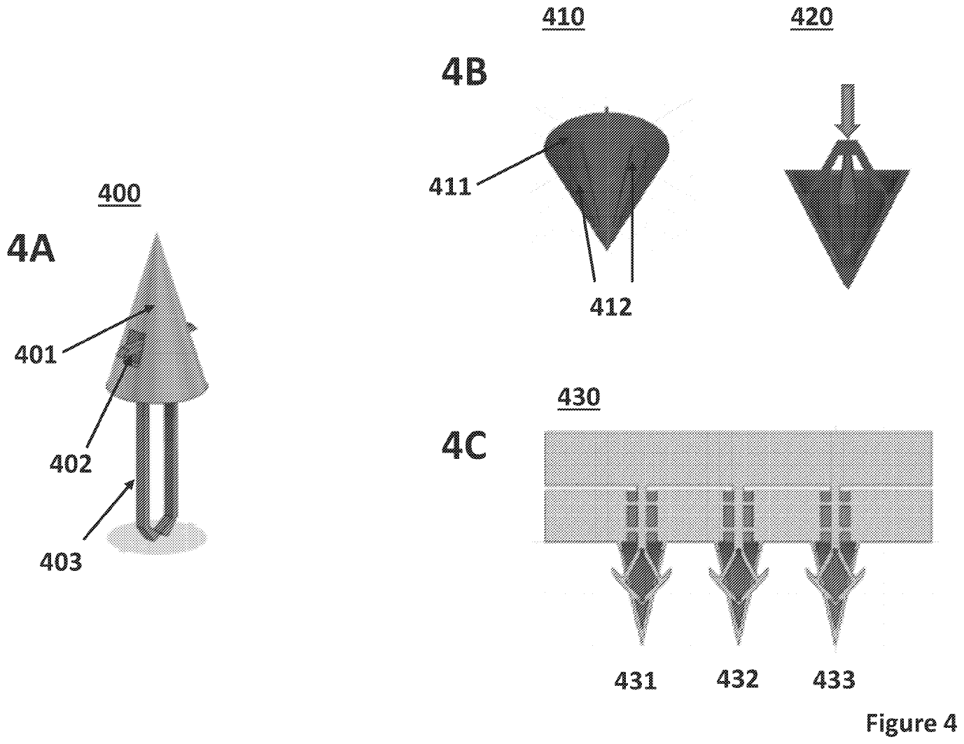

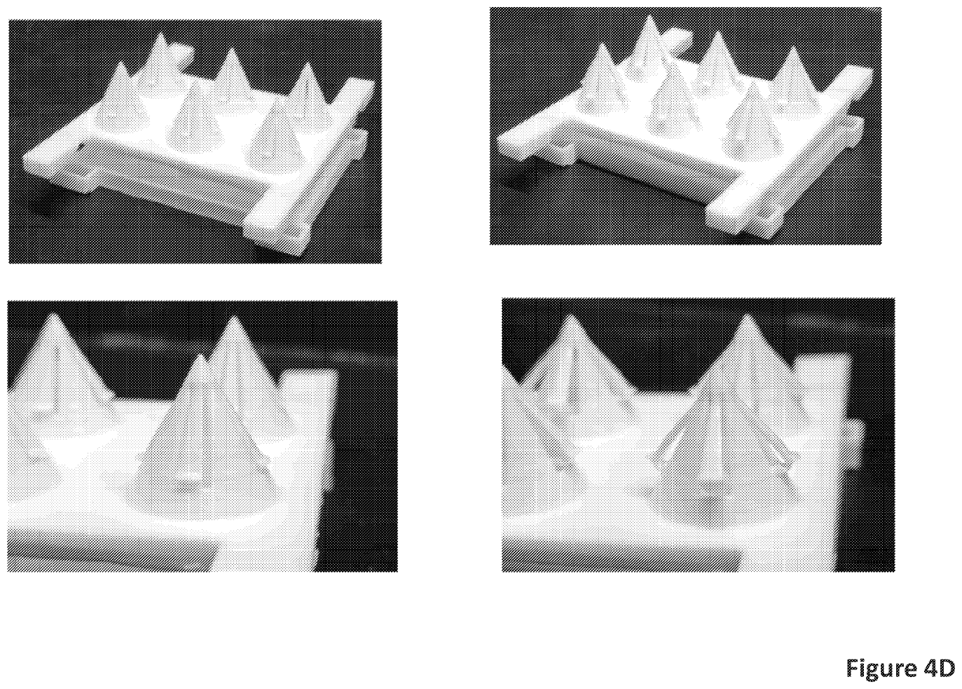

In some embodiments, one or more of the polymeric microneedles is configured to deploy a projection in response to the applied stimulus. For example, when the polymeric structure is pressed to the skin surface of a subject, a protrusion may be deployed from the microneedle. In some instances, one or more protrusions are deployed, such as 2 or more, such as 3 or more, such as 4 or more, such as 5 or more, such as 10 or more and including 12 or more protrusions are deployed in response to the applied stimulus. In some instances, the projection is a barb. In some instances, the projection is a cantilever hook. In some instances, the projection is retractable. In some embodiments, the protrusion is moved by a tweezer-like squeezing force, such as to retract the protrusion. FIG. 4A depicts a polymeric structure having a polymeric microneedle that includes a protrusion that is deployed in response to an applied stimulus according to certain embodiments. Polymeric structure 400 includes polymeric microneedle component 401 having protrusions 402 which can be deployed by applying mechanical pressure to protrusion applicators 403. Protrusions 402 are deployable and retractable by tweezer-like squeezing motion where pressure applied to protrusion applicators 403 results in motion of protrusions 402 in and out of polymeric microneedle component 401.

In some instances, each polymeric microneedle employs an umbrella-like motion where the compliant polymeric microneedle includes a retractable umbrella-like mechanism with dynamic hinges that extend when pressure is applied to the polymeric structure. In some instances, the dynamic hinges are retractable, such as when the polymeric structure is removed from a skin surface of a subject. In these embodiments, 4 or more protrusions may be deployed when the mechanical stimulus is applied, such as 8 or more, such as 12 or more and including 24 or more protrusions. FIG. 4B depicts a polymeric structure having a polymeric microneedle that includes protrusions which extend in an umbrella-like mechanism in response to an applied stimulus according to certain embodiments. Polymeric structure 410 includes polymeric microneedle component 411 having protrusions 412 which extend from the polymeric microneedle surface when pressure is applied from above (see downward pressure applied as shown in inset 420) FIG. 4C depicts a polymeric structure having a plurality of polymeric microneedles which deploy protrusions in response to applied stimulus according to certain embodiments. As shown in FIG. 4C, polymeric structure 430 includes three compliant polymeric microneedles 431, 432 and 433 which each have a set of dynamic hinges which protrude from the polymeric microneedles in an umbrella-like mechanism in response to applied mechanical pressure to the polymeric structure. FIG. 4D depicts pictures of polymeric structures having polymeric microneedles which deploy a plurality of protrusions generated by high resolution continuous liquid interface production according to certain embodiments. As shown in FIG. 4D, each polymeric microneedles includes a 4 protrusions which are configured to expand in an umbrella-like motion when mechanical pressure is applied to the polymeric structure. When no pressure is applied to the polymeric structure, the protrusions remain retracted. Application of pressure, such as when inserting into the skin surface of a subject, creates a dynamic motion to expand the protrusions.

In some embodiments, the protrusion functions to maintain the polymeric microneedle in place beneath the skin surface of the subject. In certain instances, the polymeric microneedles are configured to collect biological fluid (e.g., interstitial or dermal fluid) from the subject and retracting the protrusions closes the microneedles in order to retain the collected biological fluid within the microneedle when the polymeric structure is removed from the skin of the subject. In certain instances, the polymeric microneedles are configured to contain an active agent and deploying the protrusion by applying a stimulus thereon is sufficient to deliver the active agent compound beneath the skin surface of the subject.

In some embodiments, polymeric structures of interest include a reservoir in fluid communication with the polymeric microneedles. In some instances, the reservoir is configured to contain an active agent compound (as described in greater detail below) to be delivered transdermally to a subject. In other instances, the reservoir is configured for collecting a biological fluid from a subject, such as where interstitial or dermal fluid collected from beneath a skin surface of a subject is conveyed to the reservoir through the polymeric microneedles. Depending on the size of the polymeric structure, the reservoir may have a volume capacity of 0.001 mL or more, such as 0.005 mL or more, such as 0.01 mL or more, such as 0.05 mL or more, such as 0.1 mL or more, such as 0.5 mL or more, such as 1 mL or more, such as 2 mL or more, such as 3 mL or more, such as 4 mL or more, such as 5 mL or more, such as 6 mL or more, such as 7 mL or more, such as 8 mL or more, such as 9 mL or more, such as 10 mL or more, such as 15 mL or more, such as 20 mL or more, such as 25 mL or more and including a volume capacity of 50 mL or more. In certain instances, each polymeric microneedle is in fluidic communication with a separate reservoir which is fluidically isolated from the reservoirs of other polymeric microneedles (i.e., each polymeric microneedle has its own reservoir). In these instances, each reservoir may have a fluidic volume capacity of 0.001 mL or more, such as 0.005 mL or more, such as 0.01 mL or more, such as 0.05 mL or more, such as 0.1 mL or more, such as 0.5 mL or more, such as 1 mL or more, such as 2 mL or more, such as 3 mL or more, such as 4 mL or more, such as 5 mL or more, such as 6 mL or more, such as 7 mL or more, such as 8 mL or more, such as 9 mL or more and including 10 mL or more. In some instances, the polymeric structure includes a pump component. In certain instances, the pump is configured to draw a fluidic medium from the polymeric microneedles into the reservoir. In other instances, the pump is configured to convey a fluidic medium from the reservoir through the polymeric microneedles.

In some embodiments, one or more of the reservoir and the pump are integrated into the polymeric structure such that reservoir and pump for a single unit structure with the polymeric microneedles. In other embodiments, the polymeric structure having polymeric microneedles (such as on a substrate) are coupled to the reservoir or pump component, such as with an alignment component as described above. For example, the reservoir and pump may be coupled to the polymeric structure having the polymeric microneedles using a locking mechanism such as with notches, protrusions and holes.

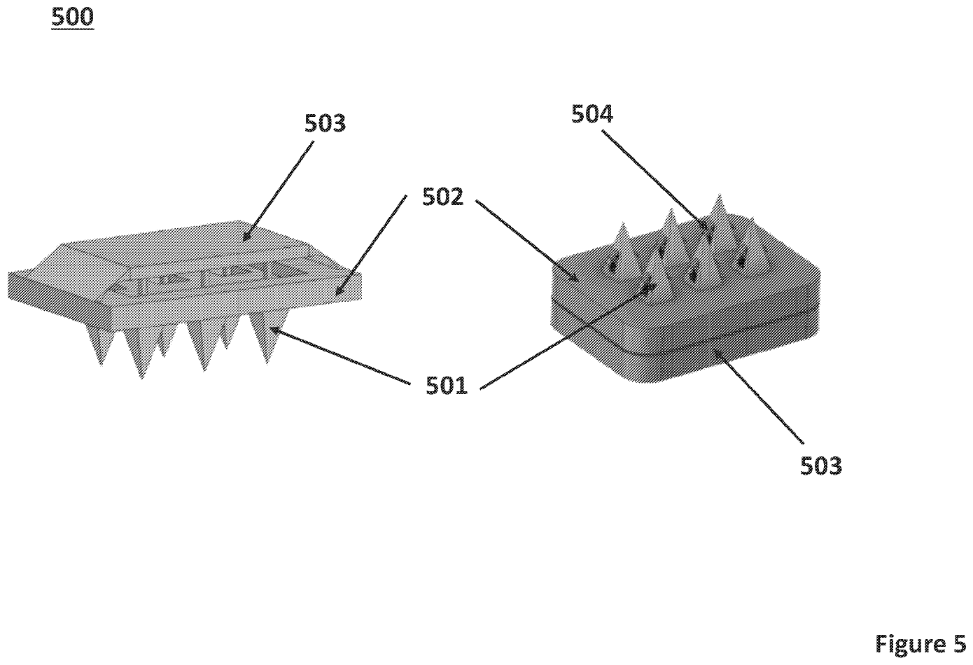

FIG. 5 depicts a polymeric structure having a pump and reservoir component according to certain embodiments. Polymeric structure 500 includes a substrate 502 having a plurality of polymeric microneedles 501 positioned thereon. In some instances, polymeric microneedles 501 include one or more protrusions 504 as described above. The polymeric structure includes a reservoir with pump component 503 integrated with the polymeric structure. In some embodiments, the reservoir contains an active agent compound. In some embodiments, the reservoir is configured to store biological fluid collected through the polymeric microneedles.

In embodiments, polymeric structures include one or more polymeric microneedles. In some instances, the polymeric structure includes a plurality of microneedles, such as 2 or more polymeric microneedles, such as 3 or more, such as 4 or more, such as 5 or more, such as 10 or more, such as 25 or more, such as 50 or more, such as 100 or more, such as 250 or more, such as 500 or more and including 1000 polymeric microneedles or more. In some instances, the polymeric structure includes an array of polymeric microneedles. In some instances, the polymeric microneedles or the array are arranged in one or more lines. For example, the polymeric microneedles may be positioned along 2 or more parallel lines, such as 3 or more, such as 4 or more, such as 5 or more, such as 6 or more, such as 7 or more, such as 8 or more, such as 9 or more, such as 10 or more, such as 15 or more, such as 20 or more and including 25 or more parallel lines of microneedles. In certain instances, the polymeric microneedles are arranged into a geometric configuration, where arrangements of interest include, but are not limited to a square configuration, rectangular configuration, trapezoidal configuration, triangular configuration, hexagonal configuration, heptagonal configuration, octagonal configuration, nonagonal configuration, decagonal configuration, dodecagonal configuration, circular configuration. oval configuration as well as irregular shaped configurations.

In some embodiments, the microneedles are separated from each other on the polymeric structure by an average distance of from 1 μm to 1000 μm, such as from 2 μm to 950 μm, such as from 3 μm to 900 μm, such as from 4 μm to 850 μm, such as from 5 μm to 800 μm, such as from 6 μm to 750 μm, such as from 7 μm to 700 μm, such as from 8 μm to 650 μm, such as from 9 μm to 600 μm, such as from 10 μm to 550 μm, such as from 15 μm to 500 μm, such as from 20 μm to 450 μm and including from 25 μm to 400 μm. The plurality of polymeric microneedles may each be the same size or patches may include plurality of polymeric microneedles having different sizes. Each polymeric microneedle independently may have a length of from 50 μm to 2000 μm, such as from 75 μm to 1950 μm, such as from 100 μm to 1900 μm, such as from 125 μm to 1850 μm, such as from 150 μm to 1800 μm, such as from 175 μm to 1750 μm, such as from 200 μm to 1700 μm, such as from 225 μm to 1650 μm, such as from 250 μm to 1600 μm, such as from 275 μm to 1550 μm and including from 300 μm to 1500 μm. Each polymeric microneedle independently may have a width (diameter when the polymeric microneedle has a circular cross-section) of from 50 μm to 1000 μm, such as from 75 μm to 950 μm, such as from 100 μm to 900 μm, such as from 125 μm to 850 μm, such as from 150 μm to 800 μm, such as from 175 μm to 750 μm, such as from 200 μm to 700 μm, such as from 225 μm to 650 μm, such as from 250 μm to 600 μm, such as from 275 μm to 550 μm and including from 300 μm to 500 μm.

In some embodiments, polymeric microneedles described herein have a lattice microstructure. In some instances, the lattice microstructures of the polymeric microneedles described herein have 2 or more repeating lattice cell units, such as 3 or more repeating lattice cell units, such as 4 or more repeating lattice cell units and including 5 or more repeating lattice cell units. In some instances, the lattice microstructure has a lattice shape selected from tetrahedral, Kagome, rhombic, icosahedral, Voronoi or triangular. In some instances, the lattice microstructure is composed of two or more lattice cell units having different lattice shapes, such where the lattice microstructure is composed of 3 or more different lattice shapes, such as 4 or more different lattice shapes and including where the lattice microstructure is composed of 5 or more different lattice shapes.

In some embodiments, the lattice microstructure is formed from lattice cells having a unit size of from 1 μm to 1000 μm, such as from 5 μm to 950 μm, such as from 10 μm to 900 μm, such as from 15 μm to 850 μm, such as from 20 μm to 800 μm, such as from 25 μm to 750 μm, such as from 30 μm to 700 μm, such as from 35 μm to 650 μm, such as from 40 μm to 600 μm, such as from 45 μm to 550 μm and including from 50 μm to 500 μm, for example from 200 μm to 500 μm. In embodiments, the lattice microstructure has a volume of from 0.01 μL to 25 μL, such as from 0.02 μL to 24.5 μL. such as from 0.03 μL to 24 μL, such as from 0.04 μL to 23.5 μL, such as rom 0.05 μL to 23 μL, such as from 0.6 μL to 22.5 μL, such as from 0.07 μL to 22 μL, such as from 0.08 μL to 21.5 μL, such as from 0.09 μL to 21 μL, such as from 0.1 μL to 20 μL, such as from 0.5 μL to 19 μL, such as from 1 μL to 18 μL, such as from 2 μL to 17 μL, such as from 3 μL to 16 μL and including from 4 μL to 15 μL. As described in greater detail below, the polymeric structure may be configured to contain a composition within the lattice microstructure (e.g., a fluidic composition) where in some embodiments the lattice microstructure is configured to contain a volume of from 0.1 μL to 25 μL, such as from 0.2 μL to 24 μL, such as from 0.3 μL to 23 μL, such as from 0.4 μL to 22 μL, such as rom 0.5 μL to 21 μL, such as from 0.6 μL to 20 μL, such as from 0.7 μL to 19 μL, such as from 0.8 μL to 18 μL, such as from 0.9 μL to 17 μL and including where the lattice microstructure is configured to contain a volume of from 1 μL to 15 μL.

In some embodiments, the density of lattice cell units remains constant throughout the lattice microstructure of polymeric structures of interest. In some instances, lattice microstructures have different densities of lattice cell units. In some instances, polymeric microneedles have a low density of lattice cell units, a medium density of lattice cell units and a high density of lattice cell units. In other embodiments, the density of lattice cell units varies at one or more parts the lattice microstructure. In some embodiments, the lattice microstructure contains regions of increased lattice cell density, such as where the lattice cell density in these regions is increased by 1% or more across the longitudinal axis of the lattice microstructure, such as by 2% or more, such as by 3% or more, such as by 4% or more, such as by 5% or more, such as by 10% or more, such as by 20% or more, such as by 30% or more, such as by 40% or more and including by 50% or more. In some instances, the regions of increased lattice cell density are present at various increments across the longitudinal axis of the lattice microstructure. For example, the regions of increased lattice cell density may be present at increments of every 10 μm or more across the longitudinal axis of the lattice microstructure, such as every 20 μm or more, such as every 30 μm or more, such as every 40 μm or more and including every 50 μm or more. In some instances, lattice microstructures have a gradient in the density of lattice cell units according to certain embodiments.

In some instances, the density of lattice cell units exhibits a gradient in one or more parts of the lattice microstructure. In certain instances, the density of lattice cell units gradually increases across a longitudinal axis of the lattice microstructure. For example, the density of the lattice cell units may increase by 1% or more across the longitudinal axis of the lattice microstructure, such as by 2% or more, such as by 3% or more, such as by 4% or more, such as by 5% or more, such as by 10% or more, such as by 20% or more, such as by 30% or more, such as by 40% or more and including by 50% or more. In some embodiments, the density of the lattice cell units increases at predetermined increments across the longitudinal axis of the lattice microstructure, such as where the density of the lattice cell units increases every 1% or more of the length across the longitudinal axis of the lattice microstructure, such as every 2% or more, such as every 3% or more, such as every 4% or more, such as every 5% or more, such as every 6% or more, such as every 7% or more, such as every 8% or more, such as every 9% or more and including every 10% or more. Depending on the size of the lattice microstructure, the density of the lattice cell units may increase every 1 μm or more across the longitudinal axis, such as every 2 μm or more, such as every 3 μm or more, such as every 4 μm or more, such as every 5 μm or more, such as every 10 μm or more, such as every 20 μm or more, such as every 30 μm or more, such as every 40 μm or more and including every 50 μm or more. For example, the density of the lattice cell units may increase by 1% or more every 25 μm or more across the longitudinal axis of the lattice microstructure, such as by 2% or more every 25 μm or more across the longitudinal axis of the lattice microstructure, such as 5% or more every 25 μm or more across the longitudinal axis of the lattice microstructure.

In some embodiments, the lattice microstructure includes a plurality of struts.

Struts according to certain embodiments provide mechanical integrity to the lattice microstructure. In some instances, struts have a thickness which range from 1 μm to 200 μm, such as from 2 μm to 190 μm, such as from 3 μm to 180 μm, such as from 4 μm to 170 μm, such as from 5 μm to 160 μm, such as from 6 μm to 150 μm, such as from 7 μm to 140 μm, such as from 8 μm to 130 μm, such as from 9 μm to 120 μm and including from 10 μm to 100 μm. For instance, the strut size may be in certain examples from 50 μm to 100 μm such as 70 μm to 90 μm. (see e.g., FIG. 3A)

In some instances, the lattice microstructures exhibit a mechanical integrity sufficient to be load bearing, such as for example as a polymeric microneedle (as described below) that can be administered to a subject. Depending on the density of the lattice microstructure, in some embodiments polymeric structures exhibit a mechanical integrity sufficient to carry a load of 0.1 N or more, such as 0.5 N or more, such as 1 N or more, such as 2 N or more, such as 3 N or more, such as 4 N or more, such as 5 N or more, such as 10 N or more, such as 15 N or more, such as 20 N or more, such as 25 N or more, such as 50 N or more, such as 75 N or more and including 100 N or more. In some embodiments, the lattice microstructure includes one or more structural support struts which is positioned within the lattice microstructure to provide increased mechanical integrity, such as where the mechanical integrity is increased by 5% or more, such as by 25% or more and including by 75% or more. For example, the structural support struts may increase the load that the lattice microstructure can carry by 0.5 N or more, such as by 1 N or more, such as by 5 N or more, such as by 10 N or more, such as by 25 N or more, such as by 50 N or more and including by 100 N or more. In some instances, the structural support struts are positioned within the interior of the lattice microstructure. In other embodiments, the support struts are positioned along the exterior of the lattice microstructure.

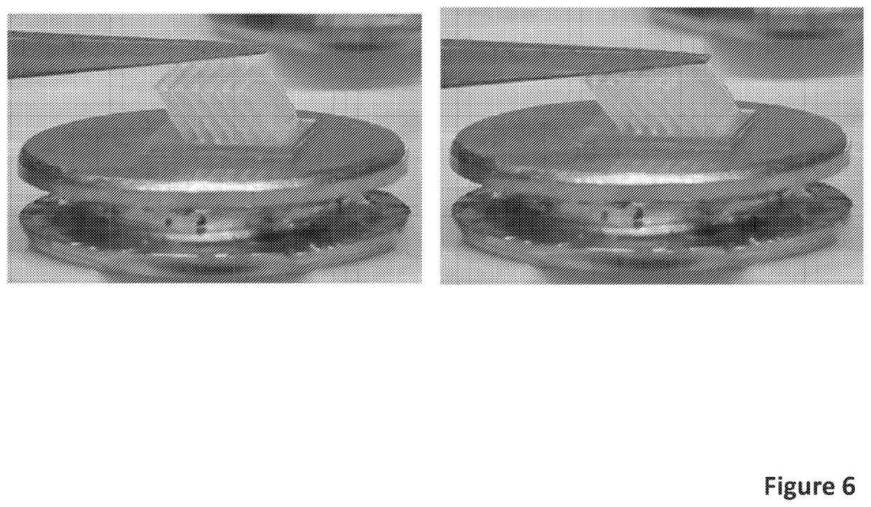

In some instances the polymeric microneedles are compliant and exhibit a flexible integrity. In some instances, the polymeric microneedles yield under a load bearing. As described above, in some embodiments, the polymeric microneedles may exhibit elastic deformation. FIG. 6 depicts a microarray of polymeric microneedles which exhibit flexibility in response to applied pressure according to certain embodiments. In some instances, the microarray of polymeric microneedles is used as a building block for micro-mechanical devices (MEMS). As shown in FIG. 6, the plurality of polymeric microneedles exhibits compliance when pressure is applied to the top of the microneedles.

Polymeric microneedles may be any three-dimensional geometric shape including but are not limited to: rectilinear cross sectional shapes, e.g., squares, rectangles, trapezoids, triangles, hexagons, etc., curvilinear cross-sectional shapes, e.g., circles, ovals, etc., as well as irregular shapes, e.g., a parabolic bottom portion coupled to a planar top portion. Polymeric structures having a lattice microstructure of interest may have a length of from 50 μm to 2000 μm, such as from 75 μm to 1950 μm, such as from 100 μm to 1900 μm, such as from 125 μm to 1850 μm, such as from 150 μm to 1800 μm, such as from 175 μm to 1750 μm, such as from 200 μm to 1700 μm, such as from 225 μm to 1650 μm, such as from 250 μm to 1600 μm, such as from 275 μm to 1550 μm and including from 300 μm to 1500 μm. Polymeric structures having a lattice microstructure of interest may have a width of from 50 μm to 1000 μm, such as from 75 μm to 950 μm, such as from 100 μm to 900 μm, such as from 125 μm to 850 μm, such as from 150 μm to 800 μm, such as from 175 μm to 750 μm, such as from 200 μm to 700 μm, such as from 225 μm to 650 μm, such as from 250 μm to 600 μm, such as from 275 μm to 550 μm and including from 300 μm to 500 μm.

In embodiments, the polymeric structure is formed from a polymerizable material which may include but is not limited to polycaprolactone, polyglycolic acid, polylactic acid, polylactic-co-glycolic acid, polyethylene glycol, thiol-enes, anhydrides, polyacrylic acid, poly methylmethacrylate, polyvinyl alcohol, polyvinylpyrrolidone, vinyl carbonates, vinyl esters, acrylamides, hyaluronic acid, chitosan, collagen, gelatin, carboxymethylcellulose. and blends or copolymers thereof. In certain embodiments, the polymeric structure is formed from polyethylene glycol dimethacrylate (PEGDMA). In certain embodiments, the polymeric structure is formed from trimethylolpropane triacrylate (TMPTA) monomer. In certain embodiments, the polymerizable material is selected from polycarbonates, polyvinyl chloride (PVC), polyurethanes, polyethers, polyamides, polyimides. or copolymers of these thermoplastics, such as PETG (glycol-modified polyethylene terephthalate), among other polymeric plastic materials. In certain embodiments, the beamsplitter is formed from a polyester, where polyesters of interest may include, but are not limited to, poly(alkylene terephthalates) such as poly(ethylene terephthalate) (PET), bottle-grade PET (a copolymer made based on monoethylene glycol, terephthalic acid, and other comonomers such as isophthalic acid, cyclohexene dimethanol, etc.), poly(butylene terephthalate) (PBT), and poly(hexamethylene terephthalate); poly(alkylene adipates) such as poly(ethylene adipate), poly(1,4-butylene adipate), and poly(hexamethylene adipate); poly(alkylene suberates) such as poly(ethylene suberate); poly(alkylene sebacates) such as poly(ethylene sebacate); poly(ε-caprolactone) and poly(β-propiolactone): poly(alkylene isophthalates) such as poly(ethylene isophthalate); poly(alkylene 2,6-naphthalene-dicarboxylates) such as poly(ethylene 2,6-naphthalene-dicarboxylate); poly(alkylene sulfonyl-4,4′-dibenzoates) such as poly(ethylene sulfonyl-4,4′-dibenzoate); poly(p-phenylene alkylene dicarboxylates) such as poly(p-phenylene ethylene dicarboxylates); poly(trans-1,4-cyclohexanediyl alkylene dicarboxylates) such as poly(trans-1,4-cyclohexanediyl ethylene dicarboxylate); poly(1,4-cyclohexane-dimethylene alkylene dicarboxylates) such as poly(1,4-cyclohexane-dimethylene ethylene dicarboxylate); poly([2.2.2]-bicyclooctane-1,4-dimethylene alkylene dicarboxylates) such as poly([2.2.2]-bicyclooctane-1,4-dimethylene ethylene dicarboxylate); lactic acid polymers and copolymers such as(S)-polylactide, (R,S)-polylactide, poly(tetramethylglycolide), and poly(lactide-co-glycolide); and polycarbonates of bisphenol A, 3,3′-dimethylbisphenol A, 3,3′,5,5′-tetrachlorobisphenol A, 3,3′,5.5′-tetramethylbisphenol A; polyamides such as poly(p-phenylene terephthalamide); polyethylene Terephthalate (e.g., Mylar™ Polyethylene Terephthalate), combinations thereof, and the like.

In some embodiments, the polymeric structures are formed from a polymerizable material which is biodegradable. The term “biodegradable” is used herein in its conventional sense to refer to a material which is capable of being decomposed, broken down or degraded by a living organism, such as microorganisms for example bacteria. In certain embodiments, the polymerizable material is dissolvable in an aqueous medium. In embodiments where the polymeric structure is formed from a dissolvable material, the lattice microstructure may be dissolved in water over a period of time of 0.01 hours or more, such as over 0.05 hours or more, such as over 0.1 hours or more, such as over 0.5 hours or more, such as over 1 hour or more, such as over 2 hours or more, such as over 6 hours or more, such as over 12 hours or more, such as over 18 hours or more, such as over 24 hours or more, such as over 36 hours or more, such as over 48 hours or more, such as over 72 hours or more, such as over 96 hours or more, such as over 120 hours or more, such as over 144 hours or more and including over 168 hours or more.

In some embodiments, the microneedle includes a tip section, a body section and a base section. In embodiments, one or more of the tip section, body section and base section of the polymeric microneedle have a lattice microstructure as described above. In some instances, one or more of the tip section, body section and base section have a solid structure (i.e., interior space that is completely filled). In some instances, one or more of the tip section, body section and base section have a hollow interior space. In certain embodiments, the microneedle includes a tip section having a solid structure, a body section having a lattice microstructure and a base section having a solid structure. In embodiments, the tip section may be a length of from 10 μm to 500 μm, such as from 20 μm to 490 μm, such as from 30 μm to 480 μm, such as from 40 μm to 470 μm, such as from 50 μm to 460 μm, such as from 60 μm to 450 μm, such as from 70 μm to 440 μm, such as from 80 μm to 430 μm, such as from 90 μm to 420 μm, such as from 100 μm to 410 μm, such as from 110 μm to 400 μm, such as from 120 μm to 390 μm, such as from 130 μm to 380 μm, such as from 140 μm to 370 μm and including from 150 μm to 360 μm. In some instances, the microneedle has a tip diameter of from 0.1 μm to 10 μm, such as from 0.5 μm to 9 μm, such as from 1 μm to 8 μm and including from 2 μm to 7 μm. In some embodiments, the body section has a length of from 10 μm to 500 μm, such as from 20 μm to 490 μm, such as from 30 μm to 480 μm, such as from 40 μm to 470 μm, such as from 50 μm to 460 μm, such as from 60 μm to 450 μm, such as from 70 μm to 440 μm, such as from 80 μm to 430 μm, such as from 90 μm to 420 μm, such as from 100 μm to 410 μm, such as from 110 μm to 400 μm, such as from 120 μm to 390 μm, such as from 130 μm to 380 μm, such as from 140 μm to 370 μm and including from 150 μm to 360 μm. In some embodiments, the base section has a length of from 10 μm to 500 μm, such as from 20 μm to 490 μm, such as from 30 μm to 480 μm, such as from 40 μm to 470 μm, such as from 50 μm to 460 μm, such as from 60 μm to 450 μm, such as from 70 μm to 440 μm, such as from 80 μm to 430 μm, such as from 90 μm to 420 μm, such as from 100 μm to 410 μm, such as from 110 μm to 400 μm, such as from 120 μm to 390 μm, such as from 130 μm to 380 μm, such as from 140 μm to 370 μm and including from 150 μm to 360 μm.

In some embodiments, the lattice microstructure of the polymeric microneedles is formed from lattice cells having a unit size of from 1 μm to 1000 μm, such as from 5 μm to 950 μm, such as from 10 μm to 900 μm, such as from 15 μm to 850 μm, such as from 20 μm to 800 μm, such as from 25 μm to 750 μm, such as from 30 μm to 700 μm, such as from 35 μm to 650 μm, such as from 40 μm to 600 μm, such as from 45 μm to 550 μm and including from 50 μm to 500 μm, for example from 200 μm to 500 μm. In embodiments, the polymeric microneedles has a volume of from 0.01 μL to 25 μL, such as from 0.02 μL to 24.5 μL, such as from 0.03 μL to 24 μL, such as from 0.04 μL to 23.5 μL, such as rom 0.05 μL to 23 μL, such as from 0.6 μL to 22.5 μL, such as from 0.07 μL to 22 μL, such as from 0.08 μL to 21.5 μL, such as from 0.09 μL to 21 μL, such as from 0.1 μL to 20 μL, such as from 0.5 μL to 19 μL, such as from 1 μL to 18 μL, such as from 2 μL to 17 μL, such as from 3 μL to 16 μL and including from 4 μL to 15 μL. In some embodiments, the polymeric microneedle is configured to deliver a volume (e.g., administering an active agent to a subject by injection) of from 0.1 μL to 25 L, such as from 0.2 μL to 24 μL, such as from 0.3 μL to 23 μL, such as from 0.4 μL to 22 μL, such as rom 0.5 μL to 21 μL, such as from 0.6 μL to 20 μL, such as from 0.7 μL to 19 μL, such as from 0.8 μL to 18 μL, such as from 0.9 μL to 17 μL and including where the lattice microstructure is configured to contain a volume of from 1 μL to 15 μL.

In certain embodiments, polymeric microneedles described herein are dynamic microneedles which can alter geometry or shape in response to an applied stimulus. In certain embodiments, the stimulus is applied mechanical pressure (e.g., pressure when inserted through a skin surface of a subject). In some instances, the dynamic microneedle is compliant and exhibits motion through elastic deformation of the polymeric microstructure. In some embodiments, the microneedle deploys a barb structure in response to the applied stimulus.

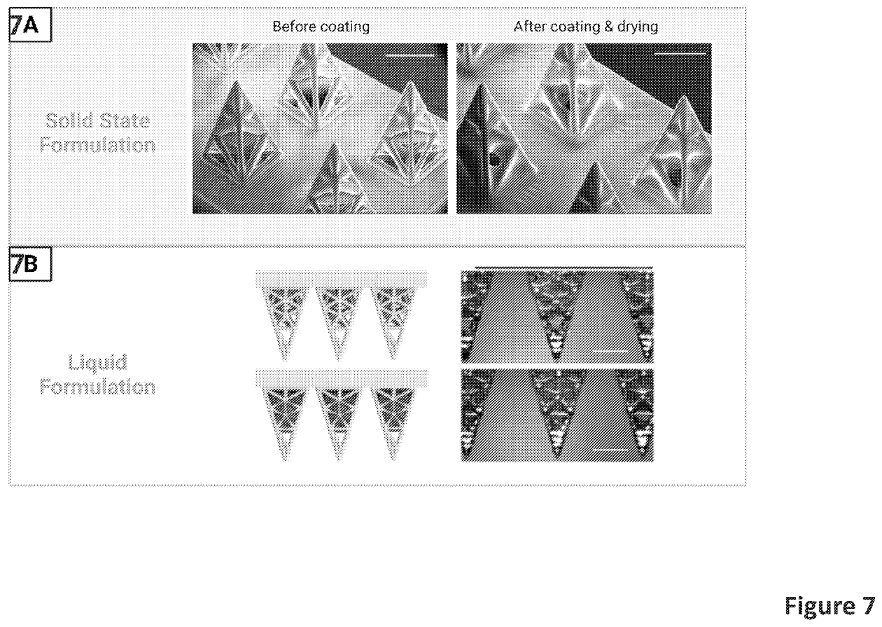

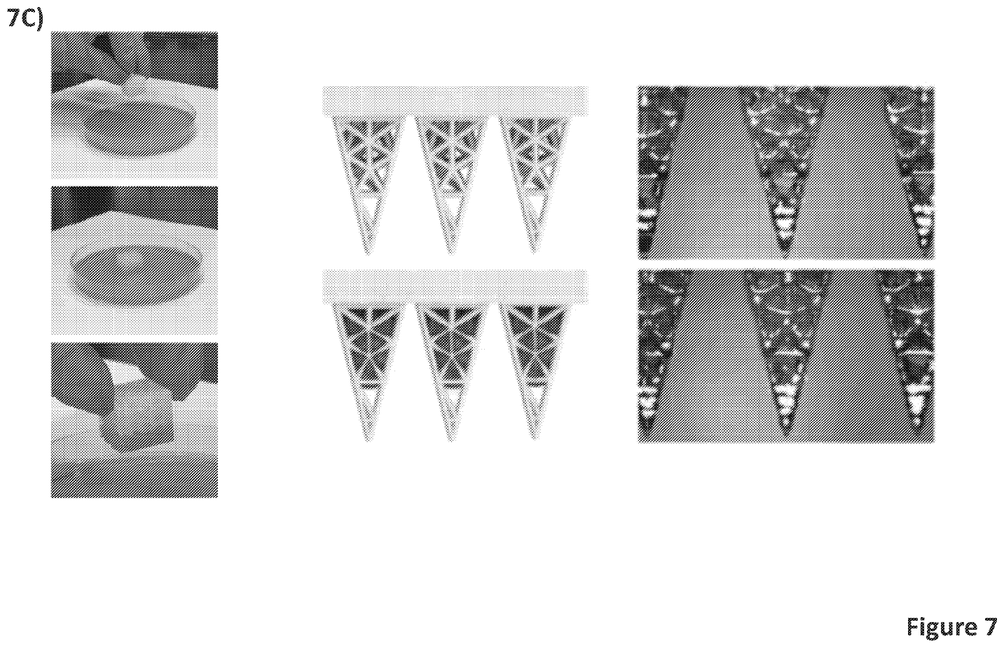

In some embodiments, polymeric microneedles also include an active agent compound. The amount of active agent compound that may be incorporated in the lattice microstructures of the polymeric microneedles described herein can vary from picogram levels to milligram levels, depending on the size of microneedles. In some embodiments, the active agent compound is a solid. In some instances where the active agent is a solid, the active is coated (e.g., by spray-coating or dip coating) onto a surface of the lattice microstructure of the polymeric microneedle. In some embodiments, the active agent compound is a liquid. In some instances where the active agent compound is a liquid, the active agent is incorporated into the lattice microstructure by liquid injection or by dip coating. In certain instances, the liquid active agent compound is incorporated into the lattice microstructure by capillary action. FIG. 7A depicts polymeric microneedles having a solid active agent compound according to certain embodiments. The solid active agent compound shown in FIG. 7A is coated onto the surface of the lattice microstructure. FIG. 7B depicts polymeric microneedles having a liquid active agent compound according to certain embodiments. The solid active agent compound shown in FIG. 7B is incorporated into the interior of the lattice microstructure by capillary action. FIG. 7C depicts capillary action by lattice microstructures according to certain embodiments. Polymeric microneedles having a lattice microstructure as described herein are placed in a colored solution. After leaving the lattice microstructure in the colored solution for a period of time, the colored solution fills the interior volume of the lattice microstructure.

Active agent compounds of interest include but are not limited to organic materials such as horseradish peroxidase, phenolsulfonphthalein, nucleotides, nucleic acids (e.g., oligonucleotides, polynucleotides, siRNA, shRNA), aptamers, antibodies or portions thereof (e.g., antibody-like molecules), hormones (e.g., insulin, testosterone), growth factors, enzymes (e.g., peroxidase, lipase, amylase, organophosphate dehydrogenase, ligases, restriction endonucleases, ribonucleases, RNA or DNA polymerases, glucose oxidase, lactase), cells (e.g., red blood cells, stem cells), bacteria or viruses, other proteins or peptides, small molecules (e.g., drugs, dyes, amino acids, vitamins, antioxidants), lipids. carbohydrates, chromophores, light emitting organic compounds (such as luciferin, carotenes) and light emitting inorganic compounds (e.g., chemical dyes and/or contrast enhancing agents such as indocyanine green), immunogenic substances such as vaccines, antibiotics, antifungal agents, antiviral agents, therapeutic agents, diagnostic agents or pro-drugs, analogs or combinations of any of the foregoing.

Examples of immunogenic vaccine substances that can be included in the microneedles described herein include, but are not limited to, those in BIOTHRAX® (anthrax vaccine adsorbed, Emergent Biosolutions, Rockville, Md.); TICE® BCG Live (Bacillus Calmette-Guerin for intravesical use, Organon Tekina Corp. LLC, Durham, N.C.); MYCOBAX® BCG Live (Sanofi Pasteur Inc); DAPTACEL® (diphtheria and tetanus toxoids and acellular pertussis [DTaP] vaccine adsorbed, Sanofi Pasteur Inc.); INFANRIX® (DTaP vaccine adsorbed, GlaxoSmithKline); TRIPEDIA® (DTaP vaccine, Sanofi Pasteur); TRIHIBIT® (DTaP/Hib, sanofi pasteur); KINRIX® (diphtheria and tetanus toxoids, acellular pertussis adsorbed and inactivated poliovirus vaccine, GlaxoSmithKline); PEDIARIX® (DTaP-HepB-IPV, GlaxoSmithKline); PENTACEL® (diphtheria and tetanus toxoids and acellular pertussis adsorbed, inactivated poliovirus and Haemophilus b conjugate [tetanus toxoid conjugate] vaccine, sanofi pasteur): Diphtheria and Tetanus Toxoids, adsorbed (for pediatric use, Sanofi Pasteur); DECAVAC® (diphtheria and tetanus toxoids adsorbed, for adult use, Sanofi Pasteur); ACTHIB® (Haemophilus b tetanus toxoid conjugate vaccine, Sanofi Pasteur); PEDVAXHIB® (Hib vaccine, Merck); Hiberix (Haemophilus b tetanus toxoid conjugate vaccine, booster dose, GlaxoSmithKline); COMVAX® (Hepatitis B-Hib vaccine, Merck); HAVRIX® (Hepatitis A vaccine, pediatric, GlaxoSmithKline); VAQTA® (Hepatitis A vaccine, pediatric, Merck); ENGERIX-B® (Hep B, pediatric, adolescent, GlaxoSmithKline); RECOMBIVAX HB® (hepatitis B vaccine, Merck); TWINRIX®, (HepA/HepB vaccine, 18 years and up, GlaxoSmithKline); CERVARIX® (human papillomavirus bivalent [types 16 and 18] vaccine, recombinant, GlaxoSmithKline); GARDASIL® (human papillomavirus bivalent [types 6, 11, 16 and 18] vaccine, recombinant, Merck); AFLURIA® (Influenza vaccine, 18 years and up, CSL); AGRIFLU™ (influenza virus vaccine for intramuscular injection, Novartis Vaccines); FLUARIX® (Influenza vaccine, 18 years and up, GlaxoSmithKline); FLULAVAL®(Influenza vaccine, 18 years and up, GlaxoSmithKline); FLUVIRIN® (Influenza vaccine, 4 years and up, Novartis Vaccine); FLUZONE® (Influenza vaccine, 6 months and up, Sanofi Pasteur); FLUMIST® (Influenza vaccine, 2 years and up, MedImmune); IPOL® (e-IPV polio vaccine, sanofi Pasteur); JE VAX® (Japanese encephalitis virus vaccine inactivated, BIKEN, Japan); IXIARO® (Japanese encephalitis virus vaccine inactivated, Novartis); MENACTRA® (Meningococcal [Groups A, C, Y and W-135] and diphtheria vaccine, Sanofi Pasteur); MENOMUNE®-A/C/Y/W-135 (Meningococcal polysaccharide vaccine, sanofi pasteur); MMRII® (MMR vaccine, Merck); MENVEO® (Meningococcal [Groups A, C, Y and W-135] oligosaccharide diphtheria CRM 197 conjugate vaccine, Novartis Vaccines); PROQUAD® (MMR and varicella vaccine, Merck); PNEUMOVAX 23® (pneumococcal polysaccharide vaccine, Merck); PREVNAR® (pneumococcal vaccine, 7-valent, Wyeth/Lederle); PREVNAR-13® (pneumococcal vaccine, 13-valent, Wyeth/Lederle); POLIO VAX™ (poliovirus inactivated, sanofi pasteur); IMOVAX® (Rabies vaccine, Sanofi Pasteur); RABAVERT™ (Rabies vaccine, Chiron); ROTATEQ® (Rotavirus vaccine, live, oral pentavalent, Merck); ROTARIX® (Rotavirus, live, oral vaccine, GlaxoSmithKline); DECAVAC™ (tetanus and diphtheria toxoids vaccine, sanofi pasteur); Td (generic) (tetanus and diphtheria toxoids, adsorbed, Massachusetts Biol. Labs); TYPHIMV1® (typhoid Vi polysaccharide vaccine, Sanofi Pasteur); ADACEL® (tetanus toxoid, reduced diphtheria toxoid and acellular pertussis, sanofi pasteur); BOOSTRIX® (tetanus toxoid, reduced diphtheria toxoid and acellular pertussis, GlaxoSmithKline); VIVOTIF® (typhoid vaccine live oral Ty21a, Bema Biotech); ACAM2000™ (Smallpox (vaccinia) vaccine, live, Acambis, Inc.); DRYVAX® (Smallpox (vaccinia) vaccine); VARIVAX® (varicella [live] vaccine, Merck); YF-VAX® (Yellow fever vaccine, Sanofi Pasteur); ZOSTAVAX®, (Varicella zoster, Merck); or combinations thereof. Any vaccine products listed in database of Center for Disease Control and Prevention (CDC) can also be included in the compositions described herein.

The term small molecule is used herein in its conventional sense to refer to natural or synthetic molecules including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. The term “antibiotic” is used herein to describe a compound that acts as an antimicrobial, bacteriostatic, or bactericidal agent. Example antibiotics include, but are not limited to, penicillins, cephalosporins, penems, carbapenems, monobactams, aminoglycosides, sulfonamides, macrolides, tetracyclins, lincosides, quinolones, chloramphenicol, vancomycin, metronidazole, rifampin, isoniazid, spectinomycin, trimethoprim. and sulfamethoxazole.

In some embodiments, the active agent compound includes but is not limited to steroids and esters of steroids (e.g., estrogen, progesterone, testosterone, androsterone, cholesterol, norethindrone, digoxigenin, cholic acid, deoxycholic acid, and chenodeoxycholic acid), boron-containing compounds (e.g., carborane), chemotherapeutic nucleotides, drugs (e.g., antibiotics, antivirals, antifungals), enediynes (e.g., calicheamicins, esperamicins, dynemicin, neocarzino statin chromophore, and kedarcidin chromophore), heavy metal complexes e.g., cisplatin), hormone antagonists (e.g., tamoxifen), non-specific (non-antibody) proteins (e.g., sugar oligomers), oligonucleotides antisense oligonucleotides that bind to a target nucleic acid sequence (e.g., mRNA sequence)), peptides. proteins, antibodies, photodynamic agents (e.g., rhodamine 123), radionuclides (e.g., 1-131, Re-186, Re-188, Y-90, Bi-212, At-211, Sr-89, Ho-166, Sm-153, Cu-67 and Cu-64). toxins (e.g., ricin), and transcription-based pharmaceuticals.

In certain embodiments, the polymeric microneedles include active agent compounds selected from acetaminophen, non-steroidal anti-inflammatory medications (NSAIDs), corticosteroids; narcotics; anti-convulsants; local anesthetics, and any combinations thereof. In various aspects of the microneedles provided herein include, but not limited to, ibuprofen, naproxin, aspirin, fenoprofen, flurbiprofen, ketoprofen, oxaprozin, diclofenac sodium, etodolac, indomethacin, ketorolac, sulindac, tolmetin, meclofenamate, mefenamic acid, nabumetone, piroxicam and COX-2 inhibitors. In some instances, the pain medications can include acetaminophen combinations (e.g., acetaminophen with a narcotic) such as acetaminophen with codeine; acetaminophen with hydrocodone; and acetaminophen with oxycodone.

In some instances, the active agent compound is coated onto one or more surfaces of the microneedle. In some instances, the active agent compound is coated onto a tip section of the microneedle. In some instances, the active agent compound is coated onto a body section of the microneedle. In some instances, the active agent compound is coated onto a base section of the microneedle. In some embodiments, the active agent compound is contained within the lattice microstructure of the polymeric microneedle. In some embodiments, the active agent compound fills 1% or more of the void volume of the lattice microstructure, such as 2% or more, such as 3% or more, such as 4% or more, such as 5% or more, such as 6% or more, such as 7% or more, such as 8% or more, such as 9% or more, such as 10% or more, such as 15% or more, such as 20% or more, such as 25% or more and including 50% or more of the void volume of the lattice microstructure. In some embodiments, each polymeric microneedle contains 0.01 μL or more of the active agent compound, such as 0.05 μL or more, such as 0.1 μL or more, such as 0.2 μL or more, such as 0.3 μL or more, such as 0.4 μL, such as 0. 5 μL or more, such as 1 μL or more, such as 2 μL or more, such as 3 μL or more, such as 4 μL or more, such as 5 μL and including 10 μL or more of the active agent compound.

In some embodiments, the polymeric microneedles are configured to release active agent compound from the lattice microstructure over a period of time of 0.01 hours or more, such as over 0.05 hours or more, such as over 0.1 hours or more, such as over 0.5 hours or more, such as over 1 hour or more, such as over 2 hours or more, such as over 6 hours or more, such as over 12 hours or more, such as over 18 hours or more, such as over 24 hours or more, such as over 36 hours or more, such as over 48 hours or more, such as over 72 hours or more, such as over 96 hours or more, such as over 120 hours or more, such as over 144 hours or more and including over 168 hours or more. In certain instances, active agent compound is released from the microneedles upon insertion or over a period of time, such as where the active agent compound is released from the microneedle over a time period of about 1 minute to about 6 months, over a time period of about 1 minute to about 3 months, over a time period of about 1 minute to about 1 month, over a time period of about 1 minute to about 2 weeks, over a time period of about 1 minute to about 1 week, over a time period of about 1 minute to about 3 days, over a time period of about 1 minute to about 1 day, over a time period of about 1 minute to about 12 hours, over a time period of about 1 minute to about 6 hours, over a time period of about 1 minute to about 1 hour, over a time period of about 1 minute to about 30 minutes, over a time period of about 30 minutes to about 6 months, over a time period of about 1 hour to about 6 months, over a time period of about 6 hours to about 6 months, over a time period of about 12 hours to about 6 months, over a time period of about 1 day to about 6 months, over a time period of about 3 days to about 6 months, over a time period of about 1 week to about 6 months, over a time period of about 2 weeks to about 6 months, over a time period of about 1 month to about 6 months, or over a time period of about 3 months to about 6 months. In certain embodiments, the active agent compound is released from the microneedle over a time period of less than about 1 minute, over a time period of about 1 second to about 1 minute, over a time period of about 1 second to about 30 seconds, over a time period of about 1 second to about 10 seconds, over a time period of about 10 seconds to about 1 minute or over a time period of about 30 seconds to about 1 minute.

In certain embodiments, the active agent compound further includes one or more excipients, such as one or more pharmaceutically acceptable excipients. In certain embodiments, the excipients include a stabilizing excipient. In certain instances, the excipient allows for dissolution of the active agent compound. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc. For example, the one or more excipients may include sucrose, starch, mannitol, sorbitol, lactose, glucose, cellulose, talc, calcium phosphate or calcium carbonate, a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

The active agent compound may be formulated into pharmaceutical compositions by combination with appropriate, pharmaceutically acceptable carriers or diluents, and may be formulated into preparations in solid, semi-solid, liquid or gaseous forms, such as powders, granules, solutions, injections, inhalants. In certain embodiments, the active agent compound is formulated for injection. For example, compositions of interest may be formulated for interstitial or dermal administration.

In pharmaceutical dosage forms, the active agent compound may be administered in the form of its pharmaceutically acceptable salts, or it may also be used alone or in appropriate association, as well as in combination, with other pharmaceutically active compounds. The following methods and excipients are merely exemplary and are in no way limiting.

In some embodiments, compositions of interest include an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. In some instances, compositions of interest further include a preservative. Suitable preservatives include, but are not limited to, a benzyl alcohol, phenol, chlorobutanol, benzalkonium chloride, and the like. In many cases, the composition is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, compositions include other additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Where the composition is formulated for injection (subcutaneous or dermal injection), the active agent compound may be formulated by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In some instances, the polymeric structures are configured for collecting a biological fluid sample from a subject. In some embodiments, the polymeric microneedle is configured to wick biological fluid into the microneedle such as through capillary action. Depending on the size of the lattice microstructure, the polymeric microneedle may be configured to collect 0.01 μL or more of the biological fluid, such as 0.05 μL or more, such as 0.1 μL or more, such as 0.2 μL or more, such as 0.3 L or more, such as 0.4 μL, such as 0. 5 μL or more, such as 1 μL or more. such as 2 μL or more, such as 3 μL or more, such as 4 μL or more, such as 5 μL and including 10 UL or more of the biological fluid. The biological fluid may be collected into the polymeric microneedle over a period of time of 1 second or more, such as 5 seconds or more, such as 10 seconds or more, such as 15 seconds or more, such as 30 seconds or more, such as 1 minute or more, such as 5 minutes or more, such as 10 minutes or more, such as 15 minutes or more, such as 30 minutes or more, such as 1 hour or more, such as 2 hours or more, such as 3 hours or more, such as 6 hours or more, such as 12 hours or more, such as 18 hours or more and including over a period of time of 24 hours or more.

In certain embodiments, polymeric structures as described above further include a backing layer. The backing layer may be flexible, such as so that it can be brought into close contact with the desired application site on the subject. The backing may be fabricated from a material that does not absorb the active agent compound or biological fluid collected from a subject into the microneedles, and does not allow the active agent compound to be leached from the interior of the lattice microstructure of the polymeric microneedles. Backing layers of interest may include, but are not limited to, non-woven fabrics, woven fabrics, films (including sheets), porous bodies, foamed bodies, paper, composite materials obtained by laminating a film on a non-woven fabric or fabric, and combinations thereof.

Non-woven fabric may include polyolefin resins such as polyethylene and polypropylene; polyester resins such as polyethylene terephthalate, polybutylene terephthalate and polyethylene naphthalate; rayon, polyamide, poly(ester ether), polyurethane, polyacrylic resins, polyvinyl alcohol, styrene-isoprene-styrene copolymers, and styrene-ethylene-propylene-styrene copolymers; and combinations thereof. Fabrics may include cotton, rayon, polyacrylic resins, polyester resins, polyvinyl alcohol, and combinations thereof. Films may include polyolefin resins such as polyethylene and polypropylene; polyacrylic resins such as polymethyl methacrylate and polyethyl methacrylate; polyester resins such as polyethylene terephthalate, polybutylene terephthalate and polyethylene naphthalate; and besides cellophane, polyvinyl alcohol, ethylene-vinyl alcohol copolymers, polyvinyl chloride, polystyrene, polyurethane, polyacrylonitrile, fluororesins, styrene-isoprene-styrene copolymers, styrene-butadiene rubber, polybutadiene, ethylene-vinyl acetate copolymers, polyamide, and polysulfone; and combinations thereof. Papers may include impregnated paper, coated paper, wood free paper, Kraft paper, Japanese paper, glassine paper, synthetic paper, and combinations thereof.

Depending on the size of the patches, the size of the backing may vary, and in some instances sized to cover the entire application site on the subject. As such, the backing layer may have a length ranging from 2 to 100 cm, such as 4 to 60 cm and a width ranging from 2 to 100 cm, such as 4 to 60 cm. In certain instances, the backing layer may insoluble in water. By insoluble in water is meant that that the backing layer may be immersed in water for a period of 1 day or longer, such as 1 week or longer, including 1 month or longer, and exhibit little if any dissolution, e.g., no observable dissolution.

In certain embodiments, patches of interest include a pressure sensitive adhesive, such as for maintaining the patch in contact with the skin surface of a subject for an extended period of time. Pressure sensitive adhesives may include, but are not limited to, poly-isobutene adhesives, poly-isobutylene adhesives, poly-isobutene/polyisobutylene adhesive mixtures, carboxylated polymers, acrylic or acrylate copolymers, such as carboxylated acrylate copolymers.

Where the pressure sensitive adhesive includes polybutene, the polybutene may be saturated polybutene. Alternatively, the polybutene may be unsaturated polybutene. Still further, the polybutene may be a mixture or combination of saturated polybutene and unsaturated polybutene. In some embodiments, the pressure sensitive adhesive may include a composition that is, or is substantially the same as, the composition of Indopol® L-2, Indopol® L-3, Indopol® L-6, Indopol® L-8, Indopol® L-14, Indopol® H-7, Indopol® H-8, Indopol® H-15, Indopol® H-25, Indopol® H-35, Indopol® H-50, Indopol® H-100, Indopol® H-300, Indopol® H-1200, Indopol® H-1500, Indopol® H-1900, Indopol® H-2100, Indopol® H-6000, Indopol® H-18000, Panalane® L-14E, Panalane® H-300E and combinations thereof. In certain embodiments, the polybutene pressure-sensitive adhesive is Indopol® H-1900. In other embodiments, the polybutene pressure-sensitive adhesive is Panalane® H-300E.

Acrylate copolymers of interest include copolymers of various monomers, such as “soft” monomers, “hard” monomers or “functional” monomers. The acrylate copolymers can be composed of a copolymer including bipolymer (i.e., made with two monomers), a terpolymer (i.e., made with three monomers), or a tetrapolymer (i.e., made with four monomers), or copolymers having greater numbers of monomers. The acrylate copolymers may be crosslinked or non-crosslinked. The polymers can be cross-linked by known methods to provide the desired polymers. The monomers from of the acrylate copolymers may include at least two or more exemplary components selected from the group including acrylic acids, alkyl acrylates, methacrylates, copolymerizable secondary monomers or monomers with functional groups. Monomers (“soft” and “hard” monomers) may be methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, acrylonitrile, methoxyethyl acrylate, methoxyethyl methacrylate, and the like. Additional examples of acrylic adhesive monomers are described in Satas, “Acrylic Adhesives,” Handbook of Pressure-Sensitive Adhesive Technology, 2nd ed., pp. 396-456 (D. Satas, ed.), Van Nostrand Reinhold, New York (1989), the disclosure of which is herein incorporated by reference. In some embodiments, the pressure sensitive adhesive is an acrylate-vinyl acetate copolymer. In some embodiments, the pressure sensitive adhesive may include a composition that is, or is substantially the same as, the composition of Duro-Tak® 87-9301, Duro-Tak® 87-200A, Duro-Tak®87-2353, Duro-Tak®87-2100, Duro-Tak®87-2051, Duro-Tak®87-2052, Duro-Tak®87-2194, Duro-Tak®87-2677, Duro-Tak®87-201A, Duro-Tak®87-2979, Duro-Tak®87-2510, Duro-Tak®87-2516, Duro-Tak®87-387, Duro-Tak®87-4287, Duro-Tak®87-2287, and Duro-Tak®87-2074 and combinations thereof. The term “substantially the same” as used herein refers to a composition that is an acrylate-vinyl acetate copolymer in an organic solvent solution. In certain embodiments, the acrylic pressure-sensitive adhesive is Duro-Tak® 87-2054.

### Methods for Applying a Polymeric Structure Having a Plurality of Polymeric Microneedles

Aspects of the present disclosure also include methods for applying the polymeric structures having a plurality of polymeric microneedles to a skin surface of a subject. In some embodiments, applying the polymeric structures described herein provide for transdermal administration of one or more active agent compounds. In some embodiments, the polymeric structures may be employed to collect a biological fluid sample by applying the patch to a skin surface of the subject. Transdermal refers to the route of administration where an active agent (i.e., drug) is delivered across the skin (e.g., topical administration) or mucous membrane or where a biological sample such as interstitial fluid is collected from the subject. As such, the polymeric structures as described herein are configured to deliver an active agent compound or collect a biological sample from the subject through one or more of the subcutis, dermis and epidermis, including the stratum corneum, stratum germinativum, stratum spinosum and stratum basale. Accordingly, the polymeric structures containing the plurality of polymeric microneedles may be applied at any convenient location, such as for example, the arms, legs, buttocks, abdomen, back, neck, scrotum, vagina, face, behind the ear, buccally as well as sublingually. In describing methods of the present invention, the term “subject” is meant the person or organism to which the patch is applied and maintained in contact. As such, subjects of the invention may include but are not limited to mammals, e.g., humans and other primates, such as chimpanzees and other apes and monkey species; and the like, where in certain embodiments the subject are humans. The term subject is also meant to include a person or organism of any age, weight or other physical characteristic, where the subjects may be an adult, a child, an infant or a newborn.

In some embodiments, methods include extended delivery of an active agent compound to the subject. By “extended delivery” is meant that the polymeric structures are configured to provide for administration of the active agent compound over an extended period of time, such as over the course of hours, days and including weeks, including 1 hour or longer, such as 2 hours or longer, such as 4 hours or longer, such as 8 hours or longer, such as 12 hours or longer, such as 24 hours or longer, such as 48 hours or longer, such as 72 hours or longer, such as 96 hours or longer, such as 120 hours or longer, such as 144 hours or longer and including 168 hours or longer. In some embodiments, the polymeric structures are configured for sustained release of the active agent compound and includes multi-day delivery of a therapeutically effective amount of the active agent compound. By multi-day delivery is meant that the polymeric microneedles of the polymeric structures are formulated to provide a therapeutically effective amount of the active agent compound to a subject when applied to the skin of a subject for a period of time that is 1 day or longer, such as 2 days or longer, such as 4 days or longer, such as 7 days or longer, such as 14 days and including 30 days or longer. In certain embodiments, the polymeric structures provide a therapeutically effective amount of the active agent compound to a subject for a period of 10 days or longer. For multi-day administration, an upper limit period of time is, in some instances, 30 days or shorter, such as 28 days or shorter, such as 21 days or shorter, such as 14 days or shorter, such as 7 days or shorter and including 3 days or shorter. In certain embodiments, multi-day delivery ranges such as from 2 days to 30 days, such as from 3 days to 28 days, such as from 4 days to 21 days, such as from 5 days to 14 days and including from 6 days to 10 days.

In certain embodiments, protocols may include multiple dosage intervals. By “multiple dosage intervals” is meant more than one polymeric structure is applied and maintained in contact with the subject in a sequential manner. As such, a polymeric structure is removed from contact with the subject and a new patch is reapplied to the subject. In practicing methods of the invention, treatment regimens may include two or more dosage intervals, such as three or more dosage intervals, such as four or more dosage intervals, such as five or more dosage intervals, including ten or more dosage intervals.

The duration between dosage intervals in a multiple dosage interval treatment protocol may vary, depending on the physiology of the subject or by the treatment protocol as determined by a health care professional. For example, the duration between dosage intervals in a multiple dosage treatment protocol may be predetermined and follow at regular intervals. As such, the time between dosage intervals may vary and may be 1 day or longer, such as 2 days or longer, such as 3 days or longer, such as 4 days or longer, such as 5 days or longer, such as 6 days or longer, such as 7 days or longer, such as 10 days or longer, including 30 days or longer. An upper limit period of time between dosage intervals is, in some instances. 30 days or shorter, such as 28 days or shorter, such as 21 days or shorter, such as 14 days or shorter, such as 7 days or shorter and including 3 days or shorter. In certain embodiments, the time between dosage intervals ranges such as from 2 days to 30 days, such as from 3 days to 28 days, such as from 4 days to 21 days, such as from 5 days to 14 days and including from 6 days to 10 days.

In certain embodiments, methods further include the step of removing the polymeric structure from contact with the subject at the conclusion of a dosage interval. For example. the polymeric structure may be removed from contact with the subject after maintaining the polymeric structure in contact with the subject for 0.5 hours or more, such as 1 hour or more, such as 2 hours or more, such as 4 hours or more, such as 8 hours or more, such as 12 hours or more, such as 24 hours or more, such as 36 hours or more, such as 48 hours or more, such as 60 hours or more, such as 72 hours or more, such as 96 hours or more, such as 120 hours or more, including 144 hours or more, and including 168 hours or more. An upper limit for the amount of time the polymeric structure is maintained in contact with a subject before removal is, in some instances, 168 hours or shorter, such as 144 hours or shorter, such as 120 hours or shorter, such as 96 hours or shorter, such as 72 hours or shorter, such as 48 hours or shorter, such as 24 hours or shorter, such as 12 hours or shorter, such as 8 hours or shorter, such as 4 hours or shorter and including 2 hours or shorter.

The location on the subject for reapplying subsequent polymeric structures in multiple dosage treatment regimens may be the same or different from the location on the subject where the previous polymeric structure was removed. For example, if a first polymeric structure is applied and maintained on the leg of the subject, one or more subsequent polymeric structures may be reapplied to the same position on the leg of the subject. On the other hand, if a first polymeric structure was applied and maintained on the leg of the subject, one or more subsequent polymeric structures may be reapplied to a different position, such as the abdomen or back of the subject. Subsequent dosages applied in multiple dosage interval regimens may have the same or different active agent compound. In certain instances, a subsequent dosage interval in a treatment regimen may contain a higher or lower concentration of active agent compound than the previous dosage interval. For example, the concentration of the active agent compound may be increased in subsequent dosage intervals by 10% or greater, such as 20% or greater, such as 50% or greater, such as 75% or greater, such as 90% or greater and including 100% or greater. An upper limit for the increase in concentration of active agent compound in subsequent dosage intervals is, in some instances, 10-fold or less, such as 5-fold or less, such as 2-fold or less, such as 1-fold or less, such as 0.5-fold or less and including 0.25-fold or less.

On the other hand, the amount of active agent compound may be decreased in subsequent dosage intervals. such as by 10% or greater, such as 20% or greater, such as 50% or greater, such as 75% or greater, such as 90% or greater and including 100% or greater. An upper limit for the decrease in amount of the active agent compound in subsequent dosage intervals is, in some instances, 10-fold or less, such as 5-fold or less, such as 2-fold or less, such as 1-fold or less, such as 0.5-fold or less and including 0.25-fold or less. In other instances, a subsequent dosage interval may contain a different active agent compound than the previous dosage interval.

In some embodiments, methods include applying one or more polymeric structures to a skin surface of a subject in a manner to collect a biological fluid sample from the subject. The biological fluid sample may be collected into the polymeric microneedles of the polymeric structures by any convenient protocol, such as for example by capillary action. In embodiments, the biological fluid sample is collected from one or more of the subcutis, dermis and epidermis, including the stratum corneum, stratum germinativum, stratum spinosum and stratum basale of the subject. In certain instances, the biological fluid sample is interstitial fluid. In certain instances, the biological fluid sample is dermal fluid. In certain instances, the biological fluid sample is blood. In some embodiments, methods include collecting a biological fluid sample from the subject (e.g., interstitial fluid, dermal fluid) for detecting an analyte present in the biological sample, such as for detecting glucose.

In some embodiments, the polymeric structure is maintained in contact with the subject for an extended period of time sufficient to collect biological fluid sample from the subject, such as over the course of hours, days and including weeks, including 1 hour or longer, such as 2 hours or longer, such as 4 hours or longer, such as 8 hours or longer, such as 12 hours or longer, such as 24 hours or longer, such as 48 hours or longer, such as 72 hours or longer, such as 96 hours or longer, such as 120 hours or longer, such as 144 hours or longer and including 168 hours or longer. In some embodiments, the polymeric microneedles are configured for multi-day collection of the biological fluid sample. By multi-day collection is meant that the polymeric microneedles of the polymeric structures are configured to continuously or in predetermined intervals collect biological sample from a subject when applied to the skin of a subject for a period of time that is 1 day or longer, such as 2 days or longer, such as 4 days or longer, such as 7 days or longer, such as 14 days and including 30 days or longer. In certain embodiments, patches are maintained in contact with the subject for a period of 10 days or longer. For multi-day collection of biological samples, an upper limit period of time is, in some instances, 30 days or shorter, such as 28 days or shorter, such as 21 days or shorter, such as 14 days or shorter, such as 7 days or shorter and including 3 days or shorter. In certain embodiments, multi-day transdermal delivery ranges such as from 2 days to 30 days, such as from 3 days to 28 days, such as from 4 days to 21 days, such as from 5 days to 14 days and including from 6 days to 10 days.

In certain embodiments, protocols may include multiple collection intervals. By “multiple collection intervals” is meant more than one polymeric structure is applied and maintained in contact with the subject in a sequential manner. As such, a patch is removed from contact with the subject and a new polymeric structure is reapplied to the subject. In practicing methods of the invention, treatment regimens may include two or more collection intervals, such as three or more collection intervals, such as four or more collection intervals, such as five or more collection intervals, including ten or more collection intervals.

The duration between collection intervals in a multiple collection interval treatment protocol may vary, depending on the physiology of the subject or by the treatment protocol as determined by a health care professional. For example, the duration between collection intervals in a multiple collection protocol may be predetermined and follow at regular intervals. As such, the time between collection intervals may vary and may be 1 day or longer, such as 2 days or longer, such as 3 days or longer, such as 4 days or longer, such as 5 days or longer, such as 6 days or longer, such as 7 days or longer, such as 10 days or longer, including 30 days or longer. An upper limit period of time between collection intervals is, in some instances, 30 days or shorter, such as 28 days or shorter, such as 21 days or shorter, such as 14 days or shorter, such as 7 days or shorter and including 3 days or shorter. In certain embodiments, the time between collection intervals ranges such as from 2 days to 30 days, such as from 3 days to 28 days, such as from 4 days to 21 days, such as from 5 days to 14 days and including from 6 days to 10 days.

In certain embodiments, methods further include the step of removing the polymeric structure from contact with the subject at the conclusion of a collection interval. For example, the polymeric structure may be removed from contact with the subject after maintaining the patch in contact with the subject for 0.5 hours or more, such as 1 hour or more, such as 2 hours or more, such as 4 hours or more, such as 8 hours or more, such as 12 hours or more, such as 24 hours or more, such as 36 hours or more, such as 48 hours or more, such as 60 hours or more, such as 72 hours or more, such as 96 hours or more, such as 120 hours or more, including 144 hours or more, and including 168 hours or more. An upper limit for the amount of time the polymeric structure is maintained in contact with a subject before removal is, in some instances, 168 hours or shorter, such as 144 hours or shorter, such as 120 hours or shorter, such as 96 hours or shorter, such as 72 hours or shorter, such as 48 hours or shorter, such as 24 hours or shorter, such as 12 hours or shorter, such as 8 hours or shorter, such as 4 hours or shorter and including 2 hours or shorter.

Polymeric structures having a plurality of polymeric microneedles according to embodiments of the invention are non-irritable to the skin of the subject at the site of application. Irritation of the skin is referred to herein in its general sense to refer to adverse effects, discoloration or damage to the skin, such as for example, redness, pain, swelling or dryness. As such, in practicing methods with the subject polymeric structures the quality of the skin remains normal and is consistent throughout the entire dosage or collection interval.

In some embodiments, skin irritation is evaluated to determine the quality and color of the skin at the application site and to determine whether any damage, pain, swelling or dryness has resulted from maintaining the polymeric structure in contact with the subject. The skin may be evaluated for irritation by any convenient protocol, such as for example using the Draize scale, as disclosed in Draize, J. H., Appraisal of the Safety of Chemicals in Foods, Drugs and Cosmetics, pp. 46-49, The Association of Food and Drug Officials of the United States: Austin, Texas, the disclosure of which is herein incorporated by reference. In particular, the skin may be evaluated at the patch application site for erythema or edema. For example, grades for erythema and edema may be assigned based on visual observation or palpation:
Erythema: 0=no visible redness; 1=very slight redness (just perceptible); 2=slight but defined redness; 3=moderately intense redness; 4=severe erythema (dark red discoloration of the skin) 5=eschar formation
Edema: 0=no visible reactions or swelling; 1=very mild edema (just perceptible swelling); 2=mild edema (corners of area are well defined due to swelling); 3=moderate edema (up to 1 mm swelling); 4=severe edema (more than 1 mm swelling).

The site of application may be evaluated for skin irritation at any time during the subject methods. In some instances, the skin is evaluated for irritation while maintaining the polymeric structure in contact with the subject by observing or palpating the skin at regular intervals, e.g., every 0.25 hours, every 0.5 hours, every 1 hour, every 2 hours, every 4 hours, every 12 hours, every 24 hours, including every 72 hours, or some other interval. For instance, the site of application may be evaluated for skin irritation while maintaining the polymeric structure in contact with the subject, such as 15 minutes after applying the polymeric structure to the subject, 30 minutes after applying the polymeric structure, 1 hour after applying the transdermal delivery device, 2 hours after applying the patch, 4 hours after applying the polymeric structure, 8 hours after applying the polymeric structure, 12 hours after applying the polymeric structure, 24 hours after applying the polymeric structure, 48 hours after applying the polymeric structure, 72 hours after applying the polymeric structure, 76 hours after applying the polymeric structure, 80 hours after applying the polymeric structure, 84 hours after applying the polymeric structure, 96 hours after applying the polymeric structure, 120 hours after applying the polymeric structure, including 168 hours after applying the polymeric structure.

Methods for Making Compliant Polymeric Structures Having a Polymeric Microneedle

Aspects of the disclosure also include methods for making a polymeric structure having one or more polymeric microneedles that is configured to exhibit a macrostructural change in response to an applied stimulus. Methods according to certain embodiments is a high resolution continuous additive processing method that includes irradiating a polymerizable composition positioned between a build elevator and a build surface to generate a polymerizable composition having a first polymerized region of the polymerizable composition in contact with the build elevator and a first non-polymerized region of the polymerizable composition in contact with the build surface; displacing the build elevator away from the build surface; irradiating the first non-polymerized region of the polymerizable composition to generate a second polymerized region of the polymerizable composition in contact with the first polymerized region and a second non-polymerized region in contact with the build surface and repeating in a manner sufficient to generate the polymeric structure. These steps are repeated in a manner sufficient to generate a polymeric structure which exhibits a macrostructural change in response to an applied stimulus. For example, the steps may be repeated 2 or more times, such as 3 or more times, such as 4 or more times, such as 5 or more times, such as 10 or more times, such as 20 or more times, such as 30 or more times, such as 40 or more times, such as 50 or more times, such as 100 or more times, such as 250 or more times, such as 500 or more times and including 1000 or more times.

In some embodiments, the polymerizable composition is irradiated with a light beam generator component of a micro-digital light projection system. In some instances, the light source is a broadband light source that emits light having wavelengths from 400 nm to 1000 nm. In some instances, the broadband light source is a halogen lamp, deuterium arc lamp, xenon arc lamp, stabilized fiber-coupled broadband light source, a broadband LED with continuous spectrum, superluminescent emitting diode, semiconductor light emitting diode. wide spectrum LED white light source, a multi-LED integrated white light source, among other broadband light sources or any combination thereof. In some instances, the light source is a narrow band light source emitting a particular wavelength or a narrow range of wavelengths. In some instances, the narrow band light sources emit light having a narrow range of wavelengths, such as for example, 50 nm or less, such as 40 nm or less, such as 30 nm or less, such as 25 nm or less, such as 20 nm or less, such as 15 nm or less, such as 10 nm or less, such as 5 nm or less, such as 2 nm or less and including light sources which emit a specific wavelength of light. In some instances, the polymerizable composition is irradiated with a narrow band light source such as a narrow wavelength LED, laser diode or a broadband light source coupled to one or more optical bandpass filters, diffraction gratings, monochromators or any combination thereof.

In certain embodiments, the light source is a stroboscopic light source and the polymerizable composition is illuminated with periodic flashes of light, such as where the polymerizable composition is irradiated at a frequency of 0.01 kHz or greater, such as 0.05 kHz or greater, such as 0.1 kHz or greater, such as 0.5 kHz or greater, such as 1 kHz or greater, such as 2.5 kHz or greater, such as 5 kHz or greater, such as 10 kHz or greater, such as 25 kHz or greater, such as 50 kHz or greater and including 100 kHz or greater. In certain instances, the polymerizable composition is irradiated with a laser, such as pulsed laser or a continuous wave laser.

In some embodiments, the polymerizable composition is in contact with the build elevator and the build surface. In some instances, methods include irradiating the polymerizable composition for 1 second or longer to bond the first polymerized region of the polymerizable composition to the build elevator, such as from 5 seconds longer, such as for 10 seconds or longer, such as for 20 seconds or longer, such as for 30 seconds or longer, such as for 1 minute or longer, such as for 5 minutes or longer and including for 10 minutes or longer.

In some embodiments, the build elevator is displaced away from the build surface after the first polymerized region of the polymerizable composition is bonded to the build elevator. In some instances, the build elevator is displaced in increments of 0.001 μm or more, such as 0.005 μm or more, such as 0.01 μm or more, such as 0.05 μm or more, such as 0.1 μm or more, such as 0.5 μm or more, such as 1 μm or more, such as 2 μm or more, such as 3 μm or more, such as 4 μm or more, such as 5 μm or more and including in increments of 10 μm or more. In certain instances, the build elevator is displaced in increments of from 0.001 μm to 20 μm, such as from 0.005 μm to 19 μm, such as from 0.01 μm to 18 μm, such as from 0.05 μm to 17 μm, such as from 0.1 μm to 16 μm, such as from 0.2 μm to 17 μm, such as from 0.3 μm to 16 μm, such as from 0.4 μm to 15 μm, such as from 0.5 μm to 14 μm, such as from 0.6 μm to 13 μm, such as from 0.7 μm to 12 μm, such as from 0.8 μm to 11 μm and including from 0.9 μm to 10 μm.

In certain instances, polymerizable composition is added to the build surface after each displacement of the build elevator away from the build surface. In some instances, the polymerizable composition is continuously added to the build surface. In other instances, the polymerizable composition is added to the build surface in discreet intervals each having a predetermined amount. In some embodiments, the polymerizable composition is selected from polycaprolactone, polyglycolic acid, polylactic acid, polylactic-co-glycolic acid, polyethylene glycol, thiol-enes, anhydrides, polyacrylic acid, poly methylmethacrylate, polyvinyl alcohol, polyvinylpyrrolidone, vinyl carbonates, vinyl esters, acrylamides, hyaluronic acid, chitosan, collagen, gelatin, carboxymethylcellulose. and blends or copolymers thereof. In certain embodiments, polymeric microneedles are formed from polyethylene glycol dimethacrylate (PEGDMA).

In some embodiments, the polymerizable composition is irradiated through build surface. In some instances, the polymerizable composition is irradiated in the presence of a polymerization inhibitor. In certain embodiments, the polymerizable composition is continuously polymerized while displacing the build elevator away from the build surface. In certain cases, the polymerization inhibitor is oxygen and the build surface is permeable to oxygen. In certain instances, polymerizing the polymerizable composition in the presence of a polymerization inhibitor such as oxygen enables continuous (i.e., not layer-by-layer) generation the lattice microstructure with a liquid “dead zone” at the interface between the build surface and the building polymeric microneedle. In some instances, the dead zone is generated because oxygen acts as a polymerization inhibitor, passing through the oxygen-permeable build surface. Photopolymerization cannot occur in the oxygen containing “dead zone” region such that this region remains fluid, and the polymerized component in contact with the build surface so that the building lattice microstructure does not physically attach to the build surface.

In some embodiments, the polymeric structures described herein having a compliant macrostructure are polymerized using high resolution continuous liquid interface production such as described in International Patent Publication No. WO 2023/049267, the disclosure of which is herein incorporated by reference. In certain embodiments, the polymerizable composition is polymerized using a liquid interface polymerization module that is a continuous liquid interface production (CLIP) system such as that described in International Patent Publication No. WO 2014/126837; U.S. Patent Publication Nos. 2018/0064920; 2017/0095972; 2021/0246252 and U.S. Patent Publication Nos. 10,155,882; 10,792,857, the disclosures of which are herein incorporated by reference. In certain embodiments, the polymeric structure is generated by injection continuous liquid interface production by conveying the polymerizable composition through a conduit into a space between a build elevator and a build surface of a liquid interface production module, such as described in International Patent Application No. PCT/US2023/15406 filed on Mar. 16, 2023, the disclosure of which is herein incorporated by reference.

In some embodiments, methods include irradiating the polymerizable composition with a micro-digital light projection system as described in detail above. In some instances, methods include determining a focal plane on the build surface using the micro-digital light projection system. In some embodiments, determining the focal plane on the build surface includes irradiating the build surface with a stroboscopic light source through the tube lens and displacing the build surface until the light is focused on the build surface through the tube lens. In certain embodiments, methods for determining the focal plane on the build surface includes irradiating build surface with the stroboscopic light source with periodic flashes of light. For example, the frequency of each light pulse may be 0.0001 kHz or greater, such as 0.0005 kHz or greater, such as 0.001 kHz or greater, such as 0.005 kHz or greater, such as 0.01 kHz or greater, such as 0.05 kHz or greater, such as 0.1 kHz or greater, such as 0.5 kHz or greater, such as 1 kHz or greater, such as 2.5 kHz or greater, such as 5 kHz or greater, such as 10 kHz or greater, such as 25 kHz or greater, such as 50 kHz or greater and including 100 KHz or greater. In certain instances, the frequency of pulsed irradiation by the light source ranges from 0.00001 kHz to 1000 kHz, such as from 0.00005 kHz to 900 kHz, such as from 0.0001 kHz to 800 kHz, such as from 0.0005 kHz to 700 kHz, such as from 0.001 kHz to 600 KHz, such as from 0.005 kHz to 500 kHz, such as from 0.01 kHz to 400 kHz, such as from 0.05 kHz to 300 kHz, such as from 0.1 KHz to 200 kHz and including from 1 kHz to 100 kHz. The duration of light irradiation for each light pulse (i.e., pulse width) may vary and may be 0.000001 ms or more, such as 0.000005 ms or more, such as 0.00001 ms or more, such as 0.00005 ms or more, such as 0.0001 ms or more, such as 0.0005 ms or more, such as 0.001 ms or more, such as 0.005 ms or more, such as 0.01 ms or more, such as 0.05 ms or more, such as 0.1 ms or more, such as 0.5 ms or more, such as 1 ms or more, such as 2 ms or more, such as 3 ms or more, such as 4 ms or more, such as 5 ms or more, such as 10 ms or more, such as 25 ms or more, such as 50 ms or more, such as 100 ms or more and including 500 ms or more. For example, the duration of light irradiation may range from 0.000001 ms to 1000 ms, such as from 0.000005 ms to 950 ms, such as from 0.00001 ms to 900 ms, such as from 0.00005 ms to 850 ms, such as from 0.0001 ms to 800 ms, such as from 0.0005 ms to 750 ms, such as from 0.001 ms to 700 ms, such as from 0.005 ms to 650 ms, such as from 0.01 ms to 600 ms, such as from 0.05 ms to 550 ms, such as from 0.1 ms to 500 ms, such as from 0.5 ms to 450 ms, such as from 1 ms to 400 ms, such as from 5 ms to 350 ms and including from 10 ms to 300 ms. In some instances, methods include irradiating the build surface with a plane of light having a projected image pattern with the stroboscopic light source.

In some instances, determining the focal plane on the build surface includes adjusting the focus of the tube lens. In some instances, the focal point of the tube lens is increased to adjust the focus onto the build surface. For example, the focal point may be increased by 1 μm or more, such as by 5 μm or more, such as by 10 μm or more, such as by 50 μm or more, such as by 100 μm or more, such as by 500 μm or more, such as by 1 mm or more, such as by 5 mm or more, such as by 10 mm or more, such as by 50 mm or more and including by 100 mm or more. In some instances, the focal point of the tube lens is decreased to adjust the focus onto the build surface. For example, the focal point may be decreased by 1 μm or more, such as by 5 μm or more, such as by 10 μm or more, such as by 50 μm or more, such as by 100 μm or more, such as by 500 μm or more, such as by 1 mm or more, such as by 5 mm or more, such as by 10 mm or more, such as by 50 mm or more and including by 100 mm or more.

In some embodiments, methods include displacing the build surface until the projected image pattern is in focus with the build surface. The build surface and build elevator may be displaced using any convenient displacement protocol, such as manually (i.e., movement of the build surface or build elevator directly by hand), with assistance by a mechanical device or by a motor actuated displacement device. For example, in some embodiments the build surface or build elevator is moved with a mechanically actuated translation stage, mechanical leadscrew assembly, mechanical slide device, mechanical lateral motion device, mechanically operated geared translation device. In other embodiments, the build surface or build elevator is moved with a motor actuated translation stage, leadscrew translation assembly, geared translation device, such as those employing a stepper motor, servo motor, brushless electric motor, brushed DC motor, micro-step drive motor, high resolution stepper motor, among other types of motors. In some instances, the build surface is displaced by 1 μm or more, such as by 5 μm or more, such as by 10 μm or more, such as by 50 μm or more, such as by 100 μm or more and including by 500 μm or more. In certain embodiments, the build surface is displaced by 400 μm or less, such as 350 μm or less. such as by 300 μm or less, such as by 250 μm or less, such as by 200 μm or less, such as by 150 μm or less, such as by 100 μm or less and including by 50 μm or less.

In some instances, methods include generating an image stack having a plurality of the projected image patterns. The image stack may include 2 or more projected image patterns, such as 3 or more, such as 4 or more, such as 5 or more, such as 10 or more and including 25 or more projected image patterns. In certain instances, methods include determining the focal plane of the build surface based on the generated image stack. In embodiments, methods as described here for generating polymeric microstructures (e.g., polymeric microneedles) having a lattice microstructure provide for a resolution of 10 μm or less, such as 5 μm or less. In certain embodiments, the subject methods provide for a resolution of from 1.0 μm to 4 μm, such as from 1.5 μm to 3.8 μm.

As described above, in some instances the polymeric microneedles of the polymeric structure include an active agent compound. Methods according to certain embodiments include preparing a polymeric structure where the one or more polymeric microneedles have an active agent compound. Methods in some instances include coating the active agent compound onto a surface of the polymeric microneedle. In some instances, the active agent compound is coated onto a surface of the polymeric microneedle as a fluidic composition. In these embodiments, the fluidic composition may be applied to the surface of the polymeric microneedle by for example, dip-coating or spray coating the active agent composition. In some embodiments, methods include coating a surface of the polymeric microneedle with a solid active agent compound such as by dry-casting a powder containing the active agent compound.

In some instances, methods include coating 5% or more of the surface of the polymeric microneedle with the active agent compound, such as 10% or more, such as 15% or more, such as 20% or more, such as 25% or more, such as 50% or more, such as 75% or more, such as 90% or more and including coating 95% or more of the surface of the polymeric microneedle. In certain instances, the entire surface of the polymeric microneedle is coated with the active agent compound. In some instances, methods include coating the active agent compound onto a tip section of the polymeric microneedle. In some instances, methods include coating the active agent onto a surface of the body section of the polymeric microneedle. In some instances, methods include coating the active agent onto a surface of a base section of the polymeric microneedle. In certain instances, the lattice microstructure component of the polymeric microneedle is coated with the active agent compound.

Depending on the dosage amount of the active agent compound desired, the amount of active agent compound coated onto the surface may vary, such as coating 0.001 μg or more onto a surface of the polymeric microneedle, such as 0.005 μg or more, such as 0.01 μg or more, such as 0.05 μg or more, such as 0.1 μg or more, such as 0.5 μg or more, such as 1 μg or more, such as 5 μg or more, such as 25 μg or more, such as 50 μg or more, such as 100 μg or more and including coating 500 μg or more of the active agent compound onto the surface of the polymeric microneedle.

In some embodiments, the active agent compound is incorporated into an interior space of the lattice microstructure of the polymeric microneedle. In some instances, methods include microfluidic injection filling of the active agent compound into the lattice microstructure of the polymeric microneedles. In other instances, methods include contacting the lattice microstructure with a composition containing the active agent compound and incorporating the active agent by capillary action. In some embodiments, the polymeric microneedles are dipped into a composition containing the active agent compound and an amount of the active agent is incorporated into the void space of the lattice microstructure by capillary action. Depending on the density of the lattice cell units in the lattice microstructure, the polymeric microneedle may be contacted with (submerged within) the active agent composition for 0.01 minutes or more, such as for 0.05 minutes or more, such as for 0.1 minutes or more, such as for 0.5 minutes or more, such as from 1 minute or more, such as for 5 minutes or more, such as for 10 minutes or more, such as for 30 minutes or more, such as for 60 minutes or more and including for 6 hours or more to take up the active agent composition into the lattice microstructure.

In some embodiments, methods include preparing polymeric microneedles where the lattice microstructure contains regions of increased concentration of the active agent compound, such as where the concentration of active agent compound in these regions increases by 1% or more across the longitudinal axis of the lattice microstructure, such as by 2% or more, such as by 3% or more, such as by 4% or more, such as by 5% or more, such as by 10% or more, such as by 20% or more, such as by 30% or more, such as by 40% or more and including by 50% or more. In some instances, the regions of increased concentrations of active agent are present at various increments across the longitudinal axis of the lattice microstructure. For example, the regions of increased active agent concentration may be present at increments of every 10 μm or more across the longitudinal axis of the lattice microstructure, such as every 20 μm or more, such as every 30 μm or more, such as every 40 μm or more and including every 50 μm or more.

In some embodiments, methods for preparing a polymeric microneedle containing an active agent compound include incorporating the active agent compound into the polymerizable composition, such that when the polymeric microneedle is formed from the polymerizable composition (e.g., by high resolution digital light projection-continuous liquid interface processing as described above) the active agent compound is present within the void space of the lattice microstructure. For example, the active agent composition may be present in the polymerizable composition at a concentration of 0.005 μg/μL or more, such as 0.01 μg/μL or more, such as 0.05 μg/μL or more, such as 0.1 μg/μL or more, such as 0.5 μg/μL or more, such as 1 μg/μL or more, such as 5 μg/μL or more, such as 25 μg/μL or more, such as 50 μg/μL or more, such as 100 μg/μL or more and including coating 500 μg/μL or more. In some instances, where the lattice microstructure has regions of increased concentration of active agent compound, methods include increasing the amount of active agent composition present in the source of the polymerizable composition while preparing the polymeric microneedle, such as by increasing the amount of active agent in the polymerizable composition by 1% or more, such as by 2% or more, such as by 5% or more, such as by 10% or more, such as by 25% or more, such as by 50% or more and including by 75% or more.

### Kits

Kits for use in practicing certain methods described herein are also provided. In certain embodiments, the kits include one or more polymeric structures containing a plurality of polymeric microneedles as described above. In certain embodiments, the kits include an adhesive overlay, such as a backing layer having a pressure sensitive adhesive. In a given kit that includes two or more of the subject polymeric structures, the polymeric structures may be individually packaged or present within a common container. In certain embodiments, kits include an active agent compound for delivering to a subject, such as a small molecule active agent or an immunogenic active agent compound (e.g., a vaccine) as described above. In certain instances, the active agent compound may be pre-loaded into the polymeric microneedles of the polymeric structure device or may be present in a separate container in the kits. In some instances, the active agent compound is pre-loaded into a reservoir component which can be coupled to the polymeric structure for delivering to a subject.

In certain embodiments, the kits will further include instructions for practicing the subject methods or means for obtaining the same (e.g., a website URL directing the user to a webpage which provides the instructions), where these instructions may be printed on a substrate, where substrate may be one or more of: a package insert, the packaging, reagent containers and the like. Yet another form of these instructions is a computer readable medium, e.g., diskette, compact disk (CD), portable flash drive, USB storage, DVD, Blu-ray disk, etc.), and the like, on which the information has been recorded. Yet another form of these instructions that may be present is a website address which may be used via the internet to access the information at a removed site.

Notwithstanding the appended claims, the disclosure is also defined by the following clauses:

1. A polymeric structure comprising one or more polymeric microneedles, wherein the polymeric structure is configured to exhibit a macrostructural change in response to an applied stimulus.

2. The polymeric structure according to 1, wherein the polymeric structure comprises a plurality of polymeric microneedles.

3. The polymeric structure according to 2, wherein the polymeric structure comprises an array of polymeric microneedles.

4. The polymeric structure according to any one of 1-3, wherein the polymeric structure is configured to change shape in response to the applied stimulus.

5. The polymeric structure according to any one of 1-3, wherein the polymeric structure exhibits elastic deformation in response to the applied stimulus.

6. The polymeric structure according to any one of 1-5, wherein the polymeric structure comprises one or more hinges configured to extend laterally in response to the applied stimulus.

7. The polymeric structure according to any one of 1-5, wherein the polymeric structure comprises a housing for each polymeric microneedle, wherein the housing comprises a kerf bend configured to expand laterally in response to the applied stimulus.

8. The polymeric structure according to any one of 1-7, wherein the polymeric structure is configured to change size in response to the applied stimulus.

9. The polymeric structure according to 8, wherein the polymeric structure is configured to compress in response to the applied stimulus.

10. The polymeric structure according to 8, wherein the polymeric structure is configured to expand in response to the applied stimulus.

11. The polymeric structure according to any one of 1-10, wherein the applied stimulus is a mechanical stimulus.

12. The polymeric structure according to any one of 1-11, wherein the polymeric structure comprises:
a substrate comprising one or more polymeric microneedles; and
an alignment component.

13. The polymeric structure according to 12, wherein one or more of the substrate and the alignment component comprises an aligner.

14. The polymeric structure according to 13, wherein the aligner comprises a notch, a groove or a hole.

15. The polymeric structure according to 13, wherein the aligner comprises a protrusion.

16. The polymeric structure according to 15, wherein the aligner comprises a polygonal-shaped protrusion.

17. The polymeric structure according to 15, wherein the aligner comprises a cantilever hook.

18. The polymeric structure according to any one of 12-17, wherein the substrate is configured to be reversibly coupled to the alignment component.

19. The polymeric structure according to any one of 12-17, wherein the substrate is configured to be irreversibly coupled to the alignment component.

20. The polymeric structure according to any one of 18-19, wherein the alignment component comprises one or more holes which when coupled to the substrate is configured for passing the polymeric microneedles therethrough.

21. The polymeric structure according to any one of 1-20, wherein one or more of the polymeric microneedles is configured to deploy a projection in response to the applied stimulus.

22. The polymeric structure according to 21, wherein the projection is a barb.

23. The polymeric structure according to 21, wherein the projection is a cantilever hook.

24. The polymeric structure according to any one of 21-23, wherein the projection is retractable.

25. The polymeric structure according any one of 1-24, wherein the one or more polymeric microneedles comprises a lattice microstructure having one or more lattice cell units.

26. The polymeric structure according to 25, wherein the microneedle comprises lattice cell units having a size of from 100 μm to 1000 μm.

27. The polymeric structure according to any one of 25-26, wherein the microneedle comprises a square pyramidal or conical projection shape.

28. The polymeric structure according to any one of 25-27, wherein the microneedle comprises:
a tip section comprising a solid structure;
a body section comprising a lattice structure; and
a base section comprising a solid structure.

29 The polymeric structure according to any one of 1-28, wherein the polymeric structure is formed from one or more polymerizable materials.

30. The polymeric structure according to any one of 1-28, wherein the polymeric structure is formed from two or more different polymerizable materials.

31. The polymeric structure according to any one of 29-30, wherein each polymerizable material is selected from the group consisting of polycaprolactone, polyglycolic acid, polylactic acid, polylactic-co-glycolic acid, polyethylene glycol, polyethylene glycol dimethacrylate (PEGDMA), thiol-enes, anhydrides, polyacrylic acid, poly methylmethacrylate, trimethylolpropane triacrylate (TMPTA) monomer, polyvinyl alcohol, polyvinylpyrrolidone, vinyl carbonates, vinyl esters, acrylamides, hyaluronic acid. chitosan, collagen, gelatin, carboxymethylcellulose, and blends or copolymers thereof.

32. The polymeric structure according to any one of 29-31, wherein the polymerizable material comprises carbon nanotubes.

33. The polymeric structure according to any one of 1-32, wherein one or more of the polymeric microneedles is formed from a biodegradable polymerizable material.

34. The polymeric structure according to any one of 1-32, wherein the polymeric microneedles are dissolvable in an aqueous medium.

35. The polymeric structure according to any one of 1-33, wherein one or more of the polymeric microneedles further comprise an active agent compound.

36. The polymeric structure according to 35, wherein the active agent compound comprises a small molecule active agent compound.

37. The polymeric structure according to 35, wherein the active agent compound comprises an immunogenic active agent compound.

38. The polymeric structure according to 37, wherein the active agent compound comprises a vaccine.

39. The polymeric structure according to any one of 1-38, wherein the polymeric structure further comprises a reservoir in fluid communication with the polymeric microneedles.

40. The polymeric structure according to 39, wherein the reservoir comprises a pump component.

41. The polymeric structure according to 40, wherein the pump is configured to draw a fluidic medium from the polymeric microneedles into the reservoir.

42. The polymeric structure according to 40, wherein the pump is configured to convey a fluidic medium from the reservoir through the polymeric microneedles.

43. The polymeric structure according to any one of 1-42, further comprising a backing layer.

44. The polymeric structure according to 43, wherein the backing layer comprises a pressure sensitive adhesive.

45. A method comprising applying to a skin surface of a subject a polymeric structure comprising one or more polymeric microneedles, wherein the polymeric structure is configured to exhibit a macrostructural change in response to an applied stimulus.

46. The method according to 45, wherein the polymeric structure comprises a plurality of polymeric microneedles.

47. The method according to 46, wherein the polymeric structure comprises an array of polymeric microneedles.

48. The method according to any one of 45-47, wherein the polymeric microneedles comprise an active agent compound and applying the polymeric structure to the skin surface of the subject is sufficient to deliver a therapeutically effective amount of the active agent compound to the subject.

49. The method according to 48, wherein the active agent compound comprises a small molecule active agent compound.

50. The method according to 48, wherein the active agent compound comprises an immunogenic active agent compound.

51. The method according to 50, wherein the active agent compound comprises a vaccine.

52. The method according to any one of 45-47, wherein the method comprises applying the polymeric structure to the skin surface of the subject in a manner sufficient to collect a biological fluid sample from the subject into the microneedles.

53. The method according to 52, wherein the biological fluid sample comprises interstitial fluid.

54. The method according to 52, wherein the biological fluid sample comprises dermal fluid.

55. The method according to any one of any one of 52-54, wherein the method comprises collecting from 0.01 μL to 250 μL of the biological fluid from the subject.

56. The method according to any one of any one of 52-54, wherein the method comprises collecting from 0.01 μL to 2 μL of the biological fluid from the subject with each of the plurality of microneedles.

57. The method according to any one of 45-56, wherein the method comprises maintaining the polymeric structure on the skin surface of the subject for an extended period of time.

58. The method according to 57, wherein the method comprises maintaining the patch on the skin surface of the subject for 6 hours or longer.

59. The method according to 57, wherein the method comprises maintaining the patch on the skin surface of the subject for 12 hours or longer.

60. The method according to 57, wherein the method comprises maintaining the patch on the skin surface of the subject for 24 hours or longer.

61. The method according to any one of 45-60, wherein the method comprises removing the polymeric structure in 15 minutes or less from the skin surface of the subject.

62. The method according to any one of 45-61, wherein the method comprises applying mechanical pressure to the polymeric structure when applying to the skin surface of the subject.

63. The method according to any one of 45-62, wherein applying the polymeric structure to the skin surface of the subject is sufficient to change the shape of the polymeric structure.

64. The method according to 63, wherein the polymeric structure exhibits elastic deformation when applying the polymeric structure to the skin surface of the subject.

65. The method according to 64, wherein the polymeric structure comprises one or more hinges configured to extend laterally when applying the polymeric structure to the skin surface of the subject.

66. The method according to 65, wherein the hinges are configured to laterally stretch the skin surface when applying the polymeric structure to the subject.

67. The method according to 64, wherein the polymeric structure comprises a housing for each polymeric microneedle, wherein the housing comprises a kerf bend configured to expand laterally when the polymeric structure is applied to the skin surface of the subject.

68. The method according to 67, wherein the housing is configured to pinch the skin surface when applying the polymeric structure to the subject.

69. The method according to any one of 45-68, wherein the polymeric structure changes size when applying to the skin surface of the subject.

70. The method according to 69, wherein the polymeric structure compresses when applying to the skin surface of the subject.

71. The method according to 69, wherein the polymeric structure expands when applying to the skin surface of the subject.

72. The method according to any one of 45-71, wherein the polymeric structure comprises:
a substrate comprising one or more polymeric microneedles; and
an alignment component.

73. The method according to 72, wherein one or more of the substrate and the alignment component comprises an aligner.

74. The method according to 73, wherein the aligner comprises a notch, a groove or a hole.

75. The method according to 73, wherein the aligner comprises a protrusion.

76. The method according to 75, wherein the aligner comprises a polygonal-shaped protrusion.

77. The method according to 75, wherein the aligner comprises a cantilever hook.

78. The method according to any one of 72-77, wherein the substrate is configured to be reversibly coupled to the alignment component.

79. The method according to any one of 72-77, wherein the substrate is configured to be irreversibly coupled to the alignment component.

80. The method according to any one of 78-79, wherein the alignment component comprises one or more holes which when coupled to the substrate is configured for passing the polymeric microneedles therethrough.

81. The method according to any one of 45-80, wherein one or more of the polymeric microneedles is configured to deploy a projection when the polymeric structure is applied to the skin surface of the subject.

82. The method according to 81, wherein the projection is a barb.

83. The method according to 81, wherein the projection is a cantilever hook.

84. The method according to any one of 81-83, wherein the projection is retractable.

85. The method according any one of 45-84, wherein the one or more polymeric microneedles comprises a lattice microstructure having one or more lattice cell units.

86. The method according to 85, wherein the microneedle comprises lattice cell units having a size of from 100 μm to 1000 μm.

87. The method according to any one of 85-86, wherein the microneedle comprises a square pyramidal or conical projection shape.

88. The method according to any one of 85-87, wherein the microneedle comprises:
a tip section comprising a solid structure;
a body section comprising a lattice structure; and
a base section comprising a solid structure.

89. The method according to any one of 45-88, wherein the polymeric structure is formed from one or more polymerizable materials.

90. The method according to any one of 45-89, wherein the polymeric structure is formed from two or more different polymerizable materials.

91. The method according to any one of 89-90, wherein each polymerizable material is selected from the group consisting of polycaprolactone, polyglycolic acid, polylactic acid, polylactic-co-glycolic acid, polyethylene glycol, polyethylene glycol dimethacrylate (PEGDMA), thiol-enes, anhydrides, polyacrylic acid, poly methylmethacrylate, trimethylolpropane triacrylate (TMPTA) monomer, polyvinyl alcohol, polyvinylpyrrolidone, vinyl carbonates, vinyl esters, acrylamides, hyaluronic acid, chitosan, collagen, gelatin, carboxymethylcellulose, and blends or copolymers thereof.

92. The method according to any one of 28-30, wherein the polymerizable material comprises carbon nanotubes.

93. The method according to any one of 45-92, wherein one or more of the polymeric microneedles is formed from a biodegradable polymerizable material.

94. The method according to 93, wherein the microneedle is dissolvable in an aqueous medium.

95. The method according to any one of 45-94, wherein the polymeric structure further comprises a reservoir in fluid communication with the polymeric microneedles.

96. The method according to 95, wherein the reservoir comprises a pump component.

97. The method according to 96, wherein the pump is configured to draw a fluidic medium from the polymeric microneedles into the reservoir.

98. The method according to 96, wherein the pump is configured to convey a fluidic medium from the reservoir through the polymeric microneedles.

99. The method according to any one of 45-98, further comprising a backing layer.

100. The method according to 99, wherein the backing layer comprises a pressure sensitive adhesive.

101. A method of making a polymeric structure comprising one or more polymeric microneedles that is configured to exhibit a macrostructural change in response to an applied stimulus, the method comprising:
a) irradiating a polymerizable composition positioned between a build elevator and a build surface to generate a polymerizable composition comprising a first polymerized region of the polymerizable composition in contact with the build elevator and a first non-polymerized region of the polymerizable composition in contact with the build surface;
b displacing the build elevator away from the build surface;
c) irradiating the first non-polymerized region of the polymerizable composition to generate a second polymerized region of the polymerizable composition in contact with the first polymerized region and a second non-polymerized region in contact with the build surface; and
d) repeating steps a)-c) in a manner sufficient to generate a polymeric structure comprising one or more polymeric microneedles.

102. The method according to 101, wherein the polymerizable composition is in contact with the build elevator and the build surface.

103. The method according to 102, wherein the method comprises irradiating the polymerizable composition for a duration sufficient to bond the first polymerized region of the polymerizable composition to the build elevator.

104. The method according to any one of 101-103, wherein the build elevator is displaced in predetermined increments of from 0.5 μm to 1.0 μm.

105. The method according to 104, wherein the method further comprises adding polymerizable composition to the build surface after each displacement of the build elevator away from the build surface.

106. The method according to any one of 101-105, wherein the polymerizable composition is irradiated through the build surface.

107. The method according to any one of 101-106, wherein the polymerizable composition is irradiated in the presence of a polymerization inhibitor.

108. The method according to any one of 101-107, wherein the polymerizable composition is continuously polymerized while displacing the build elevator away from the build surface.

109. The method according to any one of 107-108, wherein the build surface is permeable to the polymerization inhibitor.

110. The method according to 109, wherein the polymerization inhibitor is oxygen.

111. The method according to any one of 101-110, wherein the polymerizable composition is irradiated with light.

112. The method according to 111, wherein the polymerizable composition is irradiated with a micro-digital light projection system.

113. The method according to 112, wherein the micro-digital light projection system comprises:
a light beam generator component; and
a light projection monitoring component.

114. The method according to 113, wherein the light beam generator component comprises:
a light source;
a tube lens; and
one or more projection lenses.

115. The method according to any one of 112-114, wherein the light projection monitoring component comprises a photodetector.

116. The method according to 115, wherein the photodetector comprises a charge-coupled device (CCD).

117. The method according to any one of 101-116, wherein the method comprises repeating steps a)-c) in a manner sufficient to generate a polymeric structure comprising a plurality of polymeric microneedles.

118. The method according to 117, wherein the method comprises repeating steps a)-c) in a manner sufficient to generate a polymeric structure comprising an array of polymeric microneedles.

119. The method according to any one of 101-118, wherein the generated polymeric structure is configured to change shape in response to the applied stimulus.

120. The method according to any one of 101-119, wherein the generated polymeric structure exhibits elastic deformation in response to the applied stimulus.

121. The method according to any one of 101-120, wherein the generated polymeric structure comprises one or more hinges configured to extend laterally in response to the applied stimulus.

122. The method according to any one of 101-121, wherein the generated polymeric structure comprises a housing for each polymeric microneedle, wherein the housing comprises a kerf bend configured to expand laterally in response to the applied stimulus.

123. The method according to any one of 101-122, wherein the generated polymeric structure is configured to change size in response to the applied stimulus.

124. The method according to 123, wherein the generated polymeric structure is configured to compress in response to the applied stimulus.

125. The method according to 123, wherein the polymeric structure is configured to expand in response to the applied stimulus.

126. The method according to any one of 1-11, wherein the method repeating steps a)-c) in a manner sufficient to generate a polymeric structure comprising:
a substrate comprising one or more polymeric microneedles; and
an alignment component.

127. The method according to 126, wherein one or more of the substrate and the alignment component comprises an aligner.

128. The method according to 127, wherein the aligner comprises a notch, a groove or a hole.

129. The method according to 127, wherein the aligner comprises a protrusion.

130. The method according to 129, wherein the aligner comprises a polygonal-shaped protrusion.

131. The method according to 129, wherein the aligner comprises a cantilever hook.

132. The method according to any one of 126-131, wherein the substrate is configured to be reversibly coupled to the alignment component.

133. The method according to any one of 126-132, wherein the substrate is configured to be irreversibly coupled to the alignment component.

134. The method according to any one of 132-133, wherein the alignment component comprises one or more holes which when coupled to the substrate is configured for passing the polymeric microneedles therethrough.

135. The method according to any one of 101-134, wherein one or more of the polymeric microneedles is configured to deploy a projection in response to the applied stimulus.

136. The method according to 135, wherein the projection is a barb.

137. The method according to 135, wherein the projection is a cantilever hook.

138. The method according to any one of 135-137, wherein the projection is retractable.

139. The method according any one of 101-138, wherein the generated polymeric microneedles comprise a lattice microstructure having one or more lattice cell units.

140. The method according to 139, wherein the microneedle comprises lattice cell units having a size of from 100 μm to 1000 μm.

141. The method according to any one of 139-140, wherein the generated microneedle comprises a square pyramidal or conical projection shape.

142. The method according to any one of 139-141, wherein the generated microneedle comprises:
a tip section comprising a solid structure;
a body section comprising a lattice structure; and
a base section comprising a solid structure.

143. The method according to any one of 101-142, wherein the polymeric structure is formed from one or more polymerizable materials.

144. The method according to any one of 101-142, wherein the polymeric structure is formed from two or more different polymerizable materials.

145. The method according to any one of 143-144, wherein each polymerizable material is selected from the group consisting of polycaprolactone, polyglycolic acid, polylactic acid, polylactic-co-glycolic acid, polyethylene glycol, polyethylene glycol dimethacrylate (PEGDMA), thiol-enes, anhydrides, polyacrylic acid, poly methylmethacrylate, trimethylolpropane triacrylate (TMPTA) monomer, polyvinyl alcohol, polyvinylpyrrolidone, vinyl carbonates, vinyl esters, acrylamides, hyaluronic acid, chitosan, collagen, gelatin, carboxymethylcellulose, and blends or copolymers thereof.

146. The method according to any one of 143-145, wherein the polymerizable material comprises carbon nanotubes.

147. The method according to any one of 101-146, wherein one or more of the polymeric microneedles is formed from a biodegradable polymerizable material.

148. The method according to any one of 101-147, wherein the polymeric microneedles are dissolvable in an aqueous medium.

149. A kit comprising:
a polymeric structure comprising one or more polymeric microneedles, wherein the polymeric structure is configured to exhibit a macrostructural change in response to an applied stimulus; and
instructions for applying the polymeric structure to a skin surface of a subject.

150. The kit according to 149, wherein the kit comprises two or more of the polymeric structures.

151. The kit according to any one of 149-150, wherein the kit further comprises an active agent compound.

152. The kit according to 151, wherein the active agent compound comprises a small molecule active agent compound.

153. The kit according to 151, wherein the active agent compound comprises an immunogenic active agent compound.

154. The kit according to 153, wherein the active agent compound comprises a vaccine.

155. The kit according to any one of 149-150, wherein the kit comprises instructions for applying the polymeric structure to the skin surface of the subject to collect a biological fluid sample from the subject into the microneedles.

156. The kit according to 155, wherein the biological fluid sample comprises interstitial fluid.

157. The kit according to 155, wherein the biological fluid sample comprises dermal fluid.

158. The kit according to any one of 149-157, wherein the kit further comprises a backing layer.

159. The kit according to 158, wherein the backing layer comprises a pressure sensitive adhesive.

160. The kit according to any one of 149-159, wherein the polymeric structure comprises a plurality of polymeric microneedles.

161. The kit according to 160, wherein the polymeric structure comprises an array of polymeric microneedles.

162. The kit according to any one of 149-161, wherein the polymeric structure is configured to change shape in response to the applied stimulus.

163. The kit according to any one of 149-161, wherein the polymeric structure exhibits elastic deformation in response to the applied stimulus.

164. The kit according to any one of 149-163, wherein the polymeric structure comprises one or more hinges configured to extend laterally in response to the applied stimulus.

165. The kit according to any one of 149-164, wherein the polymeric structure comprises a housing for each polymeric microneedle, wherein the housing comprises a kerf bend configured to expand laterally in response to the applied stimulus.

166. The kit according to any one of 149-165, wherein the polymeric structure is configured to change size in response to the applied stimulus.

167. The kit according to 166, wherein the polymeric structure is configured to compress in response to the applied stimulus.

168. The kit according to 166, wherein the polymeric structure is configured to expand in response to the applied stimulus.

169. The kit according to any one of 149-168, wherein the applied stimulus is a mechanical stimulus.

170. The kit according to any one of 149-169, wherein the polymeric structure comprises:
a substrate comprising one or more polymeric microneedles; and
an alignment component.

171. The kit according to 170, wherein one or more of the substrate and the alignment component comprises an aligner.

172. The kit according to 171, wherein the aligner comprises a notch, a groove or a hole.

173. The kit according to 171, wherein the aligner comprises a protrusion.

174. The kit according to 173, wherein the aligner comprises a polygonal-shaped protrusion.

175. The kit according to 173, wherein the aligner comprises a cantilever hook.

176. The kit according to any one of 170-175, wherein the substrate is configured to be reversibly coupled to the alignment component.

177. The kit according to any one of 170-175, wherein the substrate is configured to be irreversibly coupled to the alignment component.

178. The kit according to any one of 176-177, wherein the alignment component comprises one or more holes which when coupled to the substrate is configured for passing the polymeric microneedles therethrough.

179. The kit according to any one of 149-178, wherein one or more of the polymeric microneedles is configured to deploy a projection in response to the applied stimulus.

180. The kit according to 179, wherein the projection is a barb.

181. The kit according to 179, wherein the projection is a cantilever hook.

182. The kit according to any one of 179-181, wherein the projection is retractable.

183. The kit according any one of 149-182, wherein the one or more polymeric microneedles comprises a lattice microstructure having one or more lattice cell units.

184. The kit according to 183, wherein the microneedle comprises lattice cell units having a size of from 100 μm to 1000 μm.

185. The kit according to any one of 183-184, wherein the microneedle comprises a square pyramidal or conical projection shape.

186. The kit according to any one of 183-185, wherein the microneedle comprises:
a tip section comprising a solid structure;
a body section comprising a lattice structure; and
a base section comprising a solid structure.

187. The kit according to any one of 149-186, wherein the polymeric structure is formed from one or more polymerizable materials.

188. The kit according to any one of 149-186, wherein the polymeric structure is formed from two or more different polymerizable materials.

189. The kit according to any one of 187-188, wherein each polymerizable material is selected from the group consisting of polycaprolactone, polyglycolic acid, polylactic acid, polylactic-co-glycolic acid, polyethylene glycol, polyethylene glycol dimethacrylate (PEGDMA), thiol-enes, anhydrides, polyacrylic acid, poly methylmethacrylate, trimethylolpropane triacrylate (TMPTA) monomer, polyvinyl alcohol, polyvinylpyrrolidone, vinyl carbonates, vinyl esters, acrylamides, hyaluronic acid, chitosan, collagen, gelatin, carboxymethylcellulose. and blends or copolymers thereof.

190. The kit according to any one of 187-189, wherein the polymerizable material comprises carbon nanotubes.

191. The kit according to any one of 149-190, wherein one or more of the polymeric microneedles is formed from a biodegradable polymerizable material.

192. The kit according to any one of 149-190, wherein the polymeric microneedles are dissolvable in an aqueous medium.

193. The kit according to any one of 149-192, wherein the polymeric structure further comprises a reservoir in fluid communication with the polymeric microneedles.

194. The kit according to 193, wherein the reservoir comprises a pump component.

195. The kit according to 194, wherein the pump is configured to draw a fluidic medium from the polymeric microneedles into the reservoir.

196. The kit according to 194, wherein the pump is configured to convey a fluidic medium from the reservoir through the polymeric microneedles.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the 10 beginning of such limitation in the claim; if such exact phrase is not used in a limitation in the claim, then 35 U.S.C. § 112(f) or 35 U.S.C. § 112(6) is not invoked.

What is claimed is:

1. A polymeric structure comprising one or more polymeric microneedles, wherein the polymeric structure is configured to exhibit a macrostructural change in response to an applied stimulus.

2. The polymeric structure according to claim 1, wherein the polymeric structure exhibits elastic deformation in response to the applied stimulus.

3. The polymeric structure according to claim 2, wherein the polymeric structure comprises one or more hinges configured to extend laterally in response to the applied stimulus.

4. The polymeric structure according to claim 2, wherein the polymeric structure comprises a housing for each polymeric microneedle, wherein the housing comprises a kerf bend configured to expand laterally in response to the applied stimulus.

5. The polymeric structure according to any one of claims 1-4, wherein the polymeric structure comprises:
a substrate comprising one or more polymeric microneedles; and
an alignment component.

6. The polymeric structure according to claim 5, wherein one or more of the substrate and the alignment component comprises an aligner.

7. The polymeric structure according to claim 6, wherein the aligner comprises a protrusion.

8. The polymeric structure according to any one of claims 1-7, wherein one or more of the polymeric microneedles is configured to deploy a projection in response to the applied stimulus.

9. The polymeric structure according to claim 8, wherein the projection is a barb or a cantilever hook.

10. The polymeric structure according to claim 8, wherein the projection is retractable.

11. The polymeric structure according to any one of claims 1-10, further comprising one or more of a reservoir and a pump component.

12. The polymeric structure according to any one of claims 1-11, wherein one or more of the polymeric microneedles further comprise an active agent compound.

13. The polymeric structure according to any one of claims 1-11, wherein one or more of the polymeric microneedles is configured for collecting a biological fluid sample from a subject.

14. A method comprising applying to a skin surface of a subject a polymeric structure comprising one or more polymeric microneedles, wherein the polymeric structure is configured to exhibit a macrostructural change in response to an applied stimulus.

15. A method of making a polymeric structure comprising one or more polymeric microneedles that is configured to exhibit a macrostructural change in response to an applied stimulus, the method comprising:
a) irradiating a polymerizable composition positioned between a build elevator and a build surface to generate a polymerizable composition comprising a first polymerized region of the polymerizable composition in contact with the build elevator and a first non-polymerized region of the polymerizable composition in contact with the build surface;
b) displacing the build elevator away from the build surface;
c) irradiating the first non-polymerized region of the polymerizable composition to generate a second polymerized region of the polymerizable composition in contact with the first polymerized region and a second non-polymerized region in contact with the build surface; and
d) repeating steps a)-c) in a manner sufficient to generate a polymeric structure comprising one or more polymeric microneedles.
